(19) United States
(12) Patent Application Publication
(10) Pub. No.: US 2021/0395351 A1
(43) Pub. Date: Dec. 23, 2021

(54) PHARMACEUTICAL COMBINATION FOR THE TREATMENT OF CANCER

(71) Applicant: MUSC FOUNDATION FOR RESEARCH DEVELOPMENT, Charleston, SC (US)

(72) Inventor: Nancy DeMore, Isle of Palms, SC (US)

(21) Appl. No.: 17/279,857

(22) PCT Filed: Sep. 27, 2019
(86) PCT No.: PCT/US2019/053651

§ 371 Date: Mar. 25, 2021

Related U.S. Application Data
(60) Provisional application No. 62/737,155, filed on Sep. 27, 2018.

(51) Int. Cl. C07K 16/18; C07K 16/28; A61K 39/395; A61K 38/17; A61P 35/00

(52) U.S. Cl. CPC C07K 16/18; C07K 16/2896; C07K 16/2818; A61K 2039/545; A61K 38/1774; A61P 35/00; A61K 39/3955

(57) ABSTRACT

Provided herein is a pharmaceutical combination comprising SFRP2 antagonist and an PD-1 antibody antagonist. The invention also provides a method for the treatment of cancer, comprising the administration of a therapeutically effective amounts of a SFRP2 antagonist and a PD-1 antagonist to a patient in need thereof.

### CROSS -REFERENCE TO RELATED APPLICATIONS

This application claims priority of U.S. Provisional Application No. 62/737,155, filed Sep. 27, 2018, the entire contents of which is hereby incorporated by reference herein.

### FIELD OF THE INVENTION

The invention is directed to a therapy for the treatment of cancer comprising the administration of a SFRP2 antagonist either as a monotherapy or in combination with a PD-1 antagonist simultaneously or sequentially to a patient in need thereof.

All publications, patents, patent applications, and other references cited in this application are incorporated herein by reference in their entirety for all purposes and to the same extent as if each individual publication, patent, patent application or other reference was specifically and individually indicated to be incorporated by reference in its entirety for all purposes. Citation of a reference herein shall not be construed as an admission that such is prior art to the present invention.

### BACKGROUND OF THE INVENTION

Wnt ligands are secreted glycoproteins that activate downstream effectors through binding to cell surface G-protein coupled transmembrane receptors, known as frizzled receptors. Activation of Wnt signaling is involved in normal embryonic development, but dysregulation of this pathway has been implicated in tumor progression for various cancers (1, 2). Secreted frizzled related proteins (SFRPs) were previously regarded as inhibitors of the canonical Wnt-beta (β)-catenin pathway (1), suggesting that SFRP2 could be a tumor suppressor. However, several additional studies have shown that SFRP2 can act as a B-catenin agonist rather than an antagonist (3-7), suggesting a role in tumor promotion.

Substantial evidence now strongly supports the contribution of SFRP2 to promoting tumor growth, in breast cancer (5, 8-11), angiosarcoma (9, 10), osteosarcoma (12), rhabdomyosarcoma (13), alveolar soft part sarcoma (14), malignant glioma (15), multiple myeloma (16), renal cell carcinoma (2), prostate cancer (17), lung cancer (18), and melanoma (19). Additionally, in vivo SFRP2 molecular imaging shows that SFRP2 expression increases proportionally with tumor size (20), and the inventors showed that a murine SFRP2 monoclonal antibody inhibits angiosarcoma and breast cancer growth in vivo (21). Furthermore, Techavichit et al showed that SFRP2 is highly overexpressed in metastatic osteosarcoma, and overexpression in low-metastatic osteosarcoma cells increased metastases in vivo, while knockdown of SFRP2 in highly metastatic osteosarcoma decreased cell migration and invasion in vitro (12). In addition to direct effects of SFRP2 on tumor cells, SFRP2 is involved in tumor angiogenesis (9, 10, 19, 22-24). Therefore, SFRP2 plays a dual role in direct activation of tumor growth and a secondary effect on activating angiogenesis.

In endothelial cells, SFRP2 activates the non-canonical Wnt/Ca² pathway, rather than the canonical β-catenin pathway, to stimulate angiogenesis (22, 24). The Wnt/Ca²⁺ pathway is mediated through activated G proteins and phospholipases. This leads to transient increases in cytoplasmic free calcium and activation of the phosphatase, calcineurin, that dephosphorylates the nuclear factor of activated T-cells (NFAT), which then translocates from the cytoplasm to the nucleus. Increasing data support a critical role of NFAT in mediating tumor growth including cell growth, survival, invasion and angiogenesis (25). NFAT proteins also have crucial roles in the development and function of the immune system, including the activation of T-cells. Specifically nuclear NFAT cooperates with other transcription factors to regulate an array of genes involved in the functions of the immune system (26) including IL2 and cyclooxygenase 2 (27).

Combination Therapy

The administration of two drugs to treat a given condition, such as a cancer, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (44). In one example, combined administration of glatiramer acetate (GA) and interferon (IFN) has been experimentally shown to abrogate the clinical effectiveness of either therapy (49). In another experiment, it was reported that the addition of prednisone in combination therapy with IFN-β antagonized its up-regulator effect (48). Thus, when two drugs are administered to treat the same condition, it is unpredictable whether each will complement, have no effect on, or interfere with, the therapeutic activity of the other in a subject.

Not only may the interaction between two drugs affect the intended therapeutic activity of each drug, but the interaction may increase the levels of toxic metabolites (44). The interaction may also heighten or lessen the side effects of each drug. Hence, upon administration of two drugs to treat a disease, it is unpredictable what change will occur in the negative side profile of each drug. In one example, the combination of natalizumab and interferon β-1a was observed to increase the risk of unanticipated side effects (47,45,46).

Additionally, it is difficult to accurately predict when the effects of the interaction between the two drugs will become manifest. For example, metabolic interactions between drugs may become apparent upon the initial administration of the second drug, after the two drugs have reached a steady-state concentration or upon discontinuation of one of the drugs (44).

Therefore, the state of the art at the time of filing is that the effects of an add-on or combination therapy of two drugs, in particular an SFRP2 antagonist together with a PD-1 antagonist, cannot be predicted with any reasonable certainty until the results of a combination study are available.

### SUMMARY OF THE INVENTION

The present invention is directed to a pharmaceutical combination, comprising a therapeutically effective amount of SFRP2, CD38, and/or PD-1 antagonist and a therapeutically effective amount of an PD-1 antagonist. The invention is also directed to a method for the treatment of cancer, comprising the simultaneous or sequential administration of a therapeutically effective amount of SFRP2, CD38, and/or PD-1 antagonist and a therapeutically effective amount of an PD-1 antagonist to a patient in need thereof. The invention is also directed to a method for the treatment of certain cancers, comprising the administration of a therapeutically effective amount of SFRP2, CD38, and/or PD-1 antagonist to a patient in need thereof.

### BRIEF DESCRIPTION OF THE DRAWINGS

The drawings described below are for illustrative purposes only and are not intended to limit the scope of the invention.

FIG. 1: GP100 reactive mouse splenic T-cells were cultured for 3 days alone, or in the presence of Hs578T (top row) or RF420 cells (bottom row), and treated for 3 days. Intensity was measured for each condition by FACS analysis. Anti-CD3 and anti-CD28 antibodies (TCR stim) were used for this experiment as a positive control. Percent suppression was calculated based on the division index method. The division index is calculated by multiplying the proliferation index by the percentage of divided cells and thus represents the division status of the entire population. The experiments were repeated thrice. A representative overlay is represented on left, while the cumulative data from all repeats is presented in the bar diagram (*p<0.01).

FIG. 2: A) FZD5 protein is present in T-cells. B-C) T-cells were treated with SFRP2 (30 nM) for 1h, and (B) nuclear and (C) cytoplasmic fractions were isolated. Samples were probed with antibodies to the indicated protein markers. D) T-cells were treated with antigen gp100 (0.87 μM) or hSFRP2 mAb (10 μM) alone or in combination for 60 min and nuclear fraction isolated. Protein levels of NFATc3 in SFRP2-treated cells were compared to those in untreated cells. E) T-cells treated with IL-2 (6,000 u/well), with or without TCR/TGFβ (5 ng/ml) for 3 days. A-C, E). Actin: loading control for cytoplasmic fraction; Histone H3 and TATA: loading controls for nuclear fractions. B-D) Densitometry was performed using ImageJ and densities were calculated by multiplying the average intensity by the surface of each band. Loading control was used to eliminate inter-sample variability. Final results were obtained by normalizing each value to untreated controls (B-D) or antigen treated sample (D).

FIG. 3. A) splenic T-cells were treated with either IL2, IL2+TCR antigen, IL2+TCR antigen+TGFb. Or IL2+TCR antigen+TGFb and hSFRP2 mAb. Protein lysates were extracted and subjected to Western blot probing for SFRP2. This shows SFRP2 increases with TCR and TGF b, which is decreased with the hSFRP2 mAb. B) NAD+concentration mouse splenocytes were treated with IL-2 (6,000 u/well), with or without TCR/TGFβ (5 ng/ml), and with or without hSFRP2 mAb (10 nM) for 3 days (n=3 per group). hSFRP2 mAb increased NAD+concentration compared to untreated control (*p=0.02). C) Number of CD38+cells (Z axis). Cells treated as above except for 36 hours. There was an increase in CD38 +cells with the addition of TCR/TGFβ, which was significantly inhibited by the hSFRP2 mAb (n=3, **p<0.001).

FIG. 4. SFRP2 mAb inhibits PD-1 in T-cells. Spleenic T-cells are treated with IL2 alone, or IL2 with TCR antigen and TGFB, or IL2 with TCR antigen and TGFB and hSFRP2 mAb. Cells were analyzed by FACS. TCR and TGFB increase PD-1 Bar graph, which is reversed with hsFRP2 mAb.

FIG. 5. A) Osteosarcoma RF420 cells were injected intravenously in C57BL6 mice. Treatments with an IgG1 control or hSFRP2 mAb (4 mg/kg every 3 days), starting 10 days after the injection of tumor cells. Three weeks later, the animals were euthanized, their lungs were resected and surface nodules were counted *: p<0.0001; n=12). B) Representative lungs with tumor metastases. C) T-cells isolated from spleens of C57BL/6 mice injected with RF420 cells and treated with IgG1 control or hSFRP2 mAb. Cells were stained with a CD38, with a fluorochrome and mean fluorescent intensity (MFI) was analyzed by FACS. Bar graph showing the measurements of fluorescence obtained from T-cells isolated from 4 different spleens for each treatment (n=4). CD38 was statistically different with hSFRP2 mAb in both splenocytes and TILs*p<0.001.

FIG. 6. RF420 mouse osteosarcoma cells were injected in the tail vein of C57BL/6 mice. Starting on day 7 mice were treated with either IgG1 control, hSFRP2 mAb, mouse PD-1 mAb, or the combination of both antibodies for 21 days. Mice were euthanized, and lungs were harvested. The number of surface metastases and micrometastases by H&E were counted in each group. There was no decrease in number of mets with PD-1 mAb treatment. There was a significant decrease in # mets with hSFRP2 as monotherapy (p<0.001), which was further increased with the combination (p<0.001).

FIG. 7: Humanized SFRP2 mAb in vitro activity. (A) Concentration-response curve EC₅₀: half-maximal effective concentration; Kd: equilibrium dissociation constant; Hill: Hill coefficient. (B) Bar graph (C-H) Bar graphs showing the effects of increasing concentrations of hSFRP2 mAb (0 to 10 μM) on apoptosis (C, F; n=8), and necrosis (D, G; n=8) proliferation (E, H; n=12), in Hs578T breast cancer cells (C-E), and SVR angiosarcoma cells (F-H). *: p<0.05; **: p<0.001. Proliferation was measured using Cyquant®, while apoptosis and necrosis were measured using Annexin V and propidium iodide. Results for apoptosis and necrosis are a compilation of 2 independent experiments containing 4 wells each, n=8). The results presented for proliferation are the compilation of 3 experiments, each containing 4 repeats (n=12).

FIG. 8: The effect of Humanized SFRP2 mAb in tumor growth in angiosarcoma and breast cancer. A) AUC: Area Under Curve; T ½: Half-life; CL: clearance; Vd: volume of distribution; Cmax: maximum serum concentration. Each data point represents the mean±SEM of the measurements of at least 3 independent samples (n=3 per time point). A-C) Day is counted from baseline date, which is 30 days from tumor inoculation.

FIG. 9: Humanized SFRP2 mAb treatment promotes apoptosis in tumors. Top Bar graph shows the increase in the number of apoptotic cells in tumors treated with hSFRP2 mAb (white bars) compared to IgG1 control treated tumors (black bars). *:p<0.05. Bottom images: Paraffin embedded SVR (upper panels) and Hs578T (lower panels) tumors were sectioned and processed for TUNEL staining. For each tumor, a total of 5 fields were photographed, the number of apoptotic cells (brown) was counted in each field, and averaged for each tumor. A total of 10 tumors per treatment (n=10) were used for the analysis.

FIG. 10: Humanized SFRP2 mAb reduces metastatic osteosarcoma growth. A) Number of lung surface nodules after treatments. B) Splenocytes and TILs were harvested from mice treated with IgG1 control and hSFRP2 mAb and subjected to flow cytometry.

FIG. 11: Combination of Humanized SFRP2 mAb and nivolumab inhibit metastatic osteosarcoma growth. A) Number of lung surface nodules after various treatments. B) Graph showing the measurements of fluorescence obtained from T-cells isolated from 4 different spleens for each treatment (n=4), *** p<0.001. Mean fluorescent intensity (MFI).

FIG. 12: SFRP2 competition ELISA using variant antibodies.

FIG. 13: SDS Page. 1 μg of purified lead hSFRP2 mAb on a 4-12% NuPAGE-SDS gel.

FIG. 14: Healthy donor T cell proliferation responses to test antibodies.

### DETAILED DESCIPTION OF THE INVENTION

It is to be understood that the terminology employed herein is for the purpose of describing particular embodiments, and is not intended to be limiting. Further, although any methods, devices and materials similar or equivalent to those described herein can be used in the practice or testing of the invention, certain methods, devices and materials are now described.

The invention provides a method of treating cancer comprising administering an amount of a SFRP2 antagonist and an amount of an PD-1 antagonist to a subject in need thereof wherein the amounts when taken together are effective to treat the subject. The invention also provides for a pharmaceutical combination, comprising an amount of a SFRP2 antagonist, such as a SFRP2 mAb, and an amount of a PD-1 antagonist, such as an anti-PD-1 antibody. In one embodiment, provided is a novel humanized SFRP2 monoclonal antibody (hSFRP2 mAb) that reduces CD38 in splenocytes and tumor infiltrating lymphocytes (TILs) in vivo, and has a superior concomitant effect with a PD-1 antibody at inhibiting tumor growth in vivo. In another embodiment, a humanized SFRP2 monoclonal antibody reduces PD-1 in lymphocytes in vitro. Thus the inventive hSFRP2 mAb affects cellular functions by inhibiting the non-canonical WNT pathway in multiple cell types.

In another embodiment, the invention provides a method for the treatment of cancer, comprising administering a therapeutically effective amount of a SFRP2 antagonist, CD38 antagonist, and/or PD-1 antagonist and a therapeutically effective amount of an PD-1 antagonist to a subject in need thereof.

In another embodiment, the invention provides a method for the treatment of cancer, comprising administering a therapeutically effective amount of a SFRP2 antagonist, CD38 antagonist, and PD-1 antagonist and a therapeutically effective amount of an PD-1 antagonist to a subject in need thereof.

In another embodiment, the invention provides a method for the treatment of cancer, comprising administering a therapeutically effective amount of a SFRP2 antagonist and/or CD38 antagonist and a therapeutically effective amount of an PD-1 antagonist to a subject in need thereof

In another embodiment, the invention provides a method for the treatment of cancer, comprising administering a therapeutically effective amount of a SFRP2 antagonist and CD38 antagonist and a therapeutically effective amount of an PD-1 antagonist to a subject in need thereof.

In another embodiment, the invention provides a method for the treatment of cancer, comprising administering a therapeutically effective amount of a SFRP2 antagonist or CD38 antagonist and a therapeutically effective amount of an PD-1 antagonist to a subject in need thereof.

In another embodiment, the invention provides a method for the treatment of cancer, comprising administering a therapeutically effective amount of a SFRP2 antagonist and a therapeutically effective amount of an PD-1 antagonist to a subject in need thereof.

In one embodiment, the SFRP2 antagonist is: (a) an antibody, or antigen binding fragment of an antibody, that specifically binds to, and inhibits activation of, an SFRP2 receptor, or (b) soluble form of an SFRP2 receptor that specifically binds to a SFRP2 ligand and inhibits the SFRP2 ligand from binding to the SFRP2 receptor.

In one embodiment, the PD-1 antagonist is: (a) an antibody, or antigen binding fragment of an antibody, that specifically binds to, and inhibits activation of, an PD-1 receptor, or (b) a soluble form of an PD-1 receptor that specifically binds to a PD-1 ligand and inhibits the PD-1 ligand from binding to the PD-1 receptor.

Sarcomas are a heterogeneous group of malignancies that includes >50 different subtypes, each with unique clinical and pathologic qualities. In general, there is a 50% mortality rate, and most cures are achieved with complete surgical resection with or without radiation therapy. The results from chemotherapeutic agents for unresectable or metastatic disease have been disappointing with minimal long-term benefit and a 5 year survival for patients with metastatic disease of only 15% (34). Doxorubicin has produced response rates of 20% to 25%. PD-1 inhibitors are recently being studied for sarcomas. In a retrospective study of 28 patients with metastatic soft tissue sarcomas treated with nivolumab, 50% of patients had partial response or stable disease(35). Although there is some activity of targeted agents in sarcoma, improved therapeutic agents, and novel combinations of therapeutics, are essential to improve response and outcome. In this study the inventors report the development of a humanized SFRP2 mAb which is not immunogenic and binds to SFRP2 with high affinity. The hSFPR2 mAb not only suppresses tumor growth as single agent in three tumor cell lines (angiosarcoma, osteosarcoma, and breast carcinoma-sarcoma), but in osteosarcoma this effect was much superior to a PD-1 inhibitor alone.

Blockade of either the PD-1 receptor or its ligand PD-L1 has improved overall survival in Phase III trials in patients with melanoma, non-small cell lung cancer, and kidney cancer. Early studies suggest that PD-1 pathway blockade may benefit a subset of patients in many other types of cancer. Nevertheless, the majority of patients fail to respond to PD-1 pathway blockade and insights into improving response rates are critically needed (36).

While the inventors and others have previously shown the role of SFRP2 on angiogenesis and tumor cells (9, 10, 19, 20, 22, 24), the inventors' present study reveals a new mechanism: SFRP2 not only stimulates NFAT in endothelial cells and tumor cells, but also in T-cells. Given that PD-1 induction following TCR stimulation of CD4 and CD8 T-cells require NFAT, as the calcineurin/NFAT pathway inhibitor cyclosporin A was able to block PD-1(37, 38), the inventors hypothesized that blocking SFRP2 would reduce exhaustion of effector T-cells and lead to a better tumor control. The inventors' data shows that while the exhaustion markers, CD5, and CD103 expression was not altered, there was a reduction in expression of non-canonical ectonucleotidase CD38, the expression of which on T-cells has also been recently shown to inversely correlate with tumor control(39). CD38 regulates antitumor T-cell response, and genetic ablation or antibody mediated targeting of CD38 on T-cells improves tumor control. Additionally, T-cells with reduced expression of CD38 were also shown to maintain high effector cytokine secretion ability and were not dysfunctional despite expressing PD-1. CD38 expression was also shown to be highly expressed on the non-reprogrammable PD1 dysfunctional T-cells with fixed chromatin state (40). Additionally, combined PD-1 and CTLA-4 blockade eradicates CD38 deficient tumors in mice, and tumor bearing mice treated with combined PD-1 and CTLA-4 blocking antibodies develop resistance through the up-regulation of CD38(41). Thus, lowering CD38 expression may rescue T-cells from tumor induced exhaustion.

Since calcium and NFAT signaling has been shown to regulate CD38 expression in various cell types(42), it is likely that its inhibition of SFRP2 led to a decreased in Ca2+/NFAT signaling in T-cells resulting in reduced CD38 expression. In B cells, NFATcl has been reported to be critical for CD38 expression(43), which led the inventors to hypothesize that the hSFRP2 mAb could be reducing CD38 via its inhibitory effect on NFATc3 in T-cells. However, the inventors' data supports that inhibition of SFRP2 along with PD-1 leads to better tumor control, likely due to targeting both reduced immunosuppression due to CD38 in T-cells and Wnt signaling pathway in tumors. Without the inventor's data, there was no reason to expect such a combination to have better tumor control.

In one embodiment, subject has increased expression of CD38 and/or PD-1 if any cells in the subject's body, for example, T-cells, have more expression of CD38 and/or PD-1 than a corresponding healthy subject or a cancer subject who does not have such increased expression.

Definitions

The articles “a” and “an” are used in this disclosure to refer to one or more than one (i.e., to at least one) of the grammatical object of the article.

A “subject” is a human, and the terms “subject” and “patient” are used interchangeably herein.

The term “treating,” with regard to a subject, encompasses, e.g., inducing inhibition, regression, or stasis of a disease or disorder; or curing, improving, or at least partially ameliorating the disorder; or alleviating, lessening, suppressing, inhibiting, reducing the severity of, eliminating or substantially eliminating, or ameliorating a symptom of the disease or disorder. “Inhibition” of disease progression or disease complication in a subject means preventing or reducing the disease progression and/or disease complication in the subject.

A “symptom” associated with cancer includes any clinical or laboratory manifestation associated with cancer and is not limited to what the subject can feel or observe.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration.

As used herein, “periodic administration” means repeated/recurrent administration separated by a period of time. The period of time between administrations is preferably consistent from time to time. Periodic administration can include administration, e.g., once daily, twice daily, three times daily, four times daily, weekly, twice weekly, three times weekly, four times a week and so on, etc.

As used herein, a “unit dose”, “unit doses” and “unit dosage form(s)” mean a single drug administration entity/entities.

As used herein, “effective” or “therapeutically effective” when referring to an amount of PD-1 antagonist and/or SFRP2 antagonist refers to the quantity of PD-1 antagonist and/or SFRP2 antagonist that is sufficient to yield a desired therapeutic response. In certain embodiments, an effective amount refers to an amount effective, at dosages and for periods of time necessary, to achieve the desired therapeutic or prophylactic result. A therapeutically effective amount of a SFRP2 antagonist and/or a PD-1/PD-L1 antagonist or inhibitor of the invention may vary according to factors such as the disease state, age, sex, and weight of the individual, and the ability of the antibody or antibodies to elicit a desired response in the individual. A therapeutically effective amount encompasses an amount in which any toxic or detrimental effects of the antibody or antibodies are outweighed by the therapeutically beneficial effects. In one embodiment, the amount of SFRP2 antagonist and the amount of PD-1 antagonist, when administered in combination are effective to treat the subject. According to certain embodiments, an antibody of the invention is administered in an amount of from 0.1 mg/kg body weight to 100 mg/kg body weight. According to other embodiments, an antibody of the invention is administered at an amount of from 0.5 mg/kg body weight to 20 mg/kg body weight. According to additional embodiments, an antibody of the invention are administered at an amount of from 1.0 mg/kg body weight to 10 mg/kg body weight.

The following abbreviations of the general terms used in the present description apply irrespectively of whether the terms in question appear alone or in combination with other groups:

Area under the curve (AUC); Bovine serum albumin (BSA); Calcium (Ca²⁺); Carboxyfluorescein succinimidyl ester (CFSE) ; Clearance (CL) ; Dissociation constant (Kd); Enzyme-linked immunosorbent assay (ELISA); Fetal Bovine System (FBS); Fluorescence-activated cell sorting (FACS); Frizzled 5 (FZDS); Humanized SFRP2 monoclonal antibody (hSFRP2 mAb); Human recombinant secreted frizzled related protein 2 (hrSFRP2); Horse-radish peroxidase (HRP); Half maximal effective concentration (EC50); Intravenous (i.v.); Intraperitoneal (i.p.); Modification of Basal Medium Eagle (DMEM); Mean fluorescence Intensity (MIF); Non compartmental analysis (NCA); Nuclear factor of activated T-cells (NFAT); Pharmacokinetic (PK); Programmed cell death protein 1 (PD-1); Secreted frizzled related protein 2 (SFRP2); T-cell receptor (TCR); Terminal half-life (T½); and Volume of distribution (Vd).

The combination of the invention may be formulated for its simultaneous, separate or sequential administration, with at least a pharmaceutically acceptable carrier, additive, adjuvant or vehicle as described herein. Thus, the combination of the two active compounds may be administered:
as a combination that is part of the same medicament formulation, the two active compounds are then administered simultaneously, or
as a combination of two units, each with one of the active substances giving rise to the possibility of simultaneous, sequential or separate administration.

As used herein, “combination” means an assemblage of reagents for use in therapy either by simultaneous or contemporaneous administration. Simultaneous administration refers to administration of an admixture (whether a true mixture, a suspension, an emulsion or other physical combination) of an PD-1 antagonist and a SFRP2, CD38, and/or PD-1 antagonist. In this case, the combination may be the admixture or separate containers of the PD-1 antagonist the SFRP2, CD38, and/or PD-1 antagonist that are combined just prior to administration. Contemporaneous administration, or concomitant administration refers to the separate administration of an PD-1 antagonist and the SFRP2, CD38, and/or PD-1 antagonist at the same time, or at times sufficiently close together that a synergistic activity relative to the activity of either an PD-1 antagonist alone the SFRP2, CD38, and/or PD-1 antagonist alone is observed or in close enough temporal proximately to allow the individual therapeutic effects of each agent to overlap.

As used herein, “add-on” or “add-on therapy” means an assemblage of reagents for use in therapy, wherein the subject receiving the therapy begins a first treatment regimen of one or more reagents prior to beginning a second treatment regimen of one or more different reagents in addition to the first treatment regimen, so that not all of the reagents used in the therapy are started at the same time. For example, adding PD-1 antagonist therapy to a patient already receiving SFRP2, CD38, and/or PD-1 antagonist therapy.

Any known PD-1 antagonist may be utilized in the practice of the invention, a broad variety of which are known and disclosed in the art. The PD-1 antagonist preferably neutralizes biological function after binding. The PD-1 antagonist is preferably a human PD-1 antagonist. Optionally, the PD-1 antagonist may be an antibody, such as a monoclonal antibody or fragment thereof; a chimeric monoclonal antibody (such as a human-murine chimeric monoclonal antibody); a fully human monoclonal antibody; a recombinant human monoclonal antibody; a humanized antibody fragment; a soluble PD-1 antagonist, including small molecule PD-1 blocking agents. Optionally, the PD-1 antagonist is a functional fragment or fusion protein comprising a functional fragment of a monoclonal antibody, such as a Fab, F(ab′)2, Fv and preferably Fab. Preferably a fragment is pegylated or encapsulated (e.g. for stability and/or sustained release). The PD-1 antagonist may also be a camelid antibody. As used herein, PD-1 antagonists include but are not limited to PD-1 receptor inhibitors.

The PD-1 antagonist may be selected, for example, from one or a combination of nivolumab, pembrolizumab, avelumab, durvalumab, cemiplimab, or atezolizumab, or functional fragment thereof.

Any known SFRP2 and/or CD38 antagonist may be utilized in the practice of the invention, a broad variety of which are known and disclosed in the art. The SFRP2 and/or CD38 antagonist preferably neutralizes biological function after binding. The SFRP2 and/or CD38 antagonist is preferably a human SFRP2 and/or CD38 antagonist. Optionally, the SFRP2 and/or CD38 antagonist may be an antibody, such as a monoclonal antibody or fragment thereof; a chimeric monoclonal antibody (such as a human-murine chimeric monoclonal antibody); a fully human monoclonal antibody; a recombinant human monoclonal antibody; a humanized antibody fragment; a soluble SFRP2 and/or CD38 antagonist, including small molecule SFRP2 and/or CD38 blocking agents. Optionally, the SFRP2 and/or CD38 antagonist is a functional fragment or fusion protein comprising a functional fragment of a monoclonal antibody, such as a Fab, F(ab′)2, Fv and preferably Fab. Preferably a fragment is pegylated or encapsulated (e.g. for stability and/or sustained release). The SFRP2 and/or CD38 antagonist may also be a camelid antibody. As used herein, SFRP2 and/or CD38 antagonists include but are not limited to SFRP2 and/or CD38 receptor inhibitors. For example, SFRP2 antagonists are disclosed in U.S. Pat. Nos. 8,734,789, and 9,073,982, the contents of which are hereby incorporated by reference.

The invention will now be further described in the Examples below, which are intended as an illustration only and do not limit the scope of the invention.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1

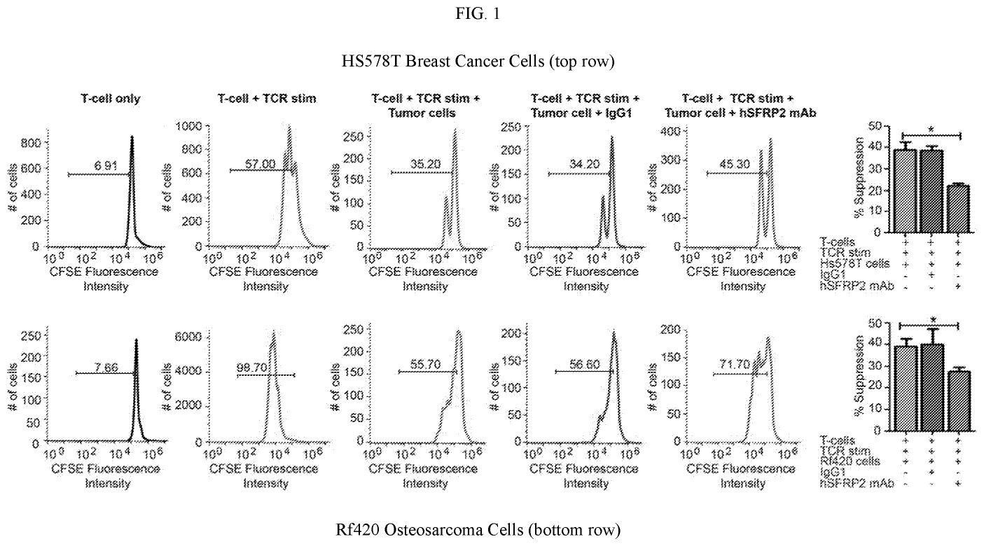

Humanized SFRP2 mAb rescues tumor cell inhibition of T-cell proliferation. Since SFRP2 activates NFATc3, and NFAT proteins regulate T-cell proliferation (28) the inventors examined whether hSFRP2 mAb affects T-cell proliferation after activation with TCR stimulation (anti-CD³/anti-CD28 antibody) (FIG. 1). T-cells were incubated alone, with TCR stimulation, with TCR stimulation+tumor cells, with TCR stimulation+tumor cells+IgG1 control, or with TCR stimulation+tumor cells+hSFRP2 mAb. Compared to the proliferation observed in T-cell alone population, TCR antigen (positive control) increased proliferation. Proliferation was reduced in the presence of Hs578T, a human metaplastic breast cancer cell line (FIG. 1). The addition of the IgG1 control to the co-culture had no effect. Comparatively, the addition of hSFRP2 mAb in the co-cultures partially rescued T-cell proliferation. This effect was also seen when T-cells were co-cultured in the presence of RF420 mouse osteosarcoma cells, where the presence of hSFRP2 mAb substantially rescued the suppression of proliferation mediated by the tumor cell (FIG. 1). Again, the addition of the IgG1 control didn't affect proliferation, compared to T-cells treated with TCR in the presence of RF420 cells.

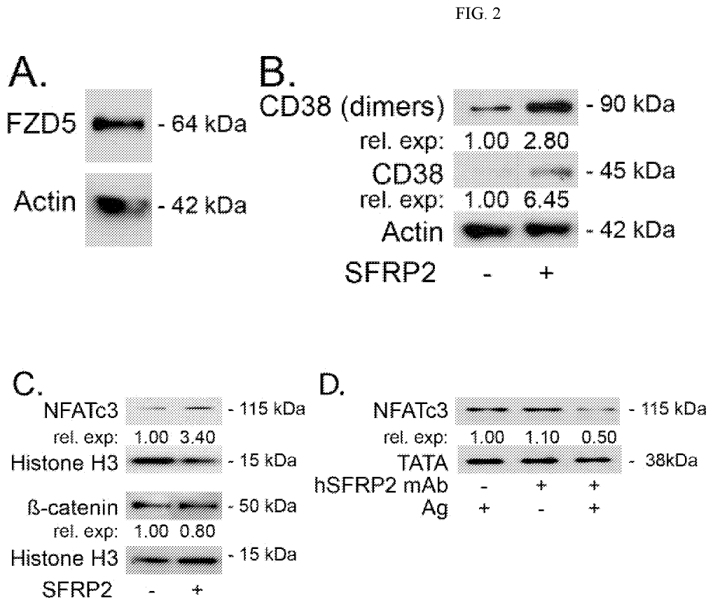

SFRP2 induces Wnt signaling in T-cells The FZD5 receptor binds SFRP2 in endothelial cells to stimulate NFATc3 activation and angiogenesis (23). However, the role of SFRP2 in T-cell activation and Wnt signaling has not previously been evaluated. Western blot analysis of T-cell lysates showed that the FZD5 protein is present in T-cells (FIG. 2A). Mouse splenic T-cells were stimulated with SFRP2 (30 nM) for 1 hour and nuclear and cytoplasmic fraction were isolated. In the cytoplasmic fraction there was an increase in CD38 with SFRP2 treatment (FIG. 2B). In the nuclear fraction there was an increase in NFATc3 with SFRP2 treatment (FIG. 2C). Next T-cells were treated with cognate antigen for three days with or without hSFRP2 mAb, and nuclear fractions were collected. There was an increase in NFATc3 in the nuclear fraction when stimulated with cognate antigen, and NFATc3 was decreased in the nuclear fraction with hSFRP2 mAb treatment (FIG. 2D).

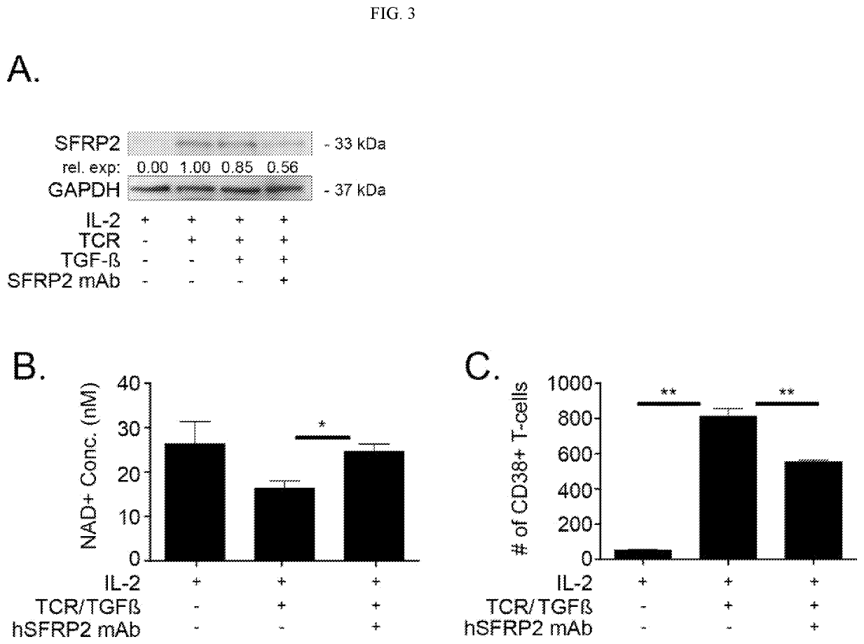

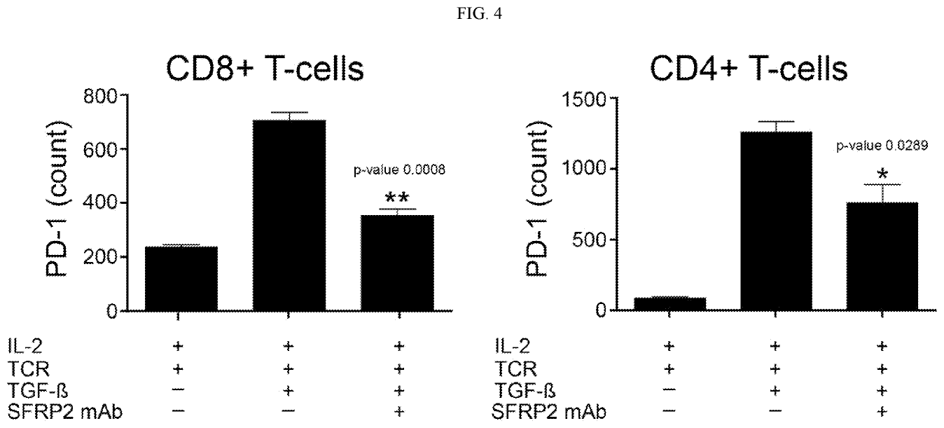

hSFRP2 mAb inhibits PD-1 and CD38 in T cells and restores NAD. Next, it was evaluated whether hSFRP2 mAb treatment of T-cells in vitro inhibits CD38 and restores NAD+levels in TGFβ-exposed T-cells. TGFβ is a cytokine present in the tumor microenvironment that increases CD38 from T-cells. FIG. 3A shows treatment of splenic T-cells with IL2, TCR antigen, and TGFβ results in an increase in SFRP2 by western blot. FACS analysis showed a statistically significant increase in CD38 +cells with the addition of TCR/TGFβ, which was significantly inhibited by the hSFRP2 mAb (FIG. 3c, n=3, p<0.001). Along with this there was an inverse increase in NAD+concentration with hSFRP2 treatment (FIG. 3b, n=3, p=0.02). Further, it was considered whether the SFRP2 mAb directly inhibited PD-1 in splenic T-cells. CD8+ and CD4+ splenic T-cells were treated with IL2, TCR antigen and TGFβ, which increase PD-1. This was reversed with the addition of the SFRP2 mAb (FIG. 4).

### Example 2

Osteosarcoma Prognosis and Treatment Options. Osteosarcoma (OS) is the most common primary malignancy of bone, usually affecting adolescents and young adults. If feasible, the primary tumor is resected surgically, with both neoadjuvant chemotherapy and adjuvant chemotherapy delivered. However even with chemotherapy, only two-thirds of patients with initially resectable disease are cured, with long-term survival occurring in <30% of patients with metastatic or recurrent tumors. The lung is involved in about 80% of cases with metastatic disease and subsequent respiratory distress is responsible for most of the fatalities (29). Although immunotherapy has shown efficacy in some tumor types, administration of pembrolizumab resulted in a lack of efficacy in the treatment of osteosarcoma in a phase II trial (SARCO28), in which only 5% of patients with metastatic osteosarcoma had an objective response to pembrolizumab(30). The lack of new active agents has blocked any progress in increasing survival of osteosarcoma patients for over three decades, and novel treatment approaches are greatly needed (31).

Growing evidence strongly supports the contribution of secreted SFRP2 to osteosarcoma metastases. SFRP2 is overexpressed in metastatic osteosarcoma compared to non-metastatic osteosarcoma (32). High expression of SFRP2 in OS patient samples correlates with poor survival and SFRP2 overexpression suppresses normal osteoblast differentiation, promotes OS features, and facilitates angiogenesis (33). Functional studies revealed stable overexpression of SFRP2 within localized human and mouse OS cells significantly increased cell migration and invasive ability in vitro and enhanced metastatic potential in vivo. Additional studies knocking down SFRP2 within metastatic OS cells showed a decreased cell migration and invasion ability in vitro, thus corroborating a critical biological phenotype carried out by SFRP2 (12). Thus, SFRP2 has emerged as a potential therapeutic target for osteosarcoma. SFRP2 has also been shown to contribute to tumor growth in breast cancer (5, 8-11), angiosarcoma (9, 10), rhabdomyosarcoma (13), alveolar soft part sarcoma (14), malignant glioma (15), multiple myeloma (16), renal cell carcinoma (2), prostate cancer (17), lung cancer (18), and melanoma (19). Given the lack of efficacy of immunotherapy in osteosarcoma and the inventor's data detailed elsewhere in this application, the inventors investigated whether the combination of a humanized SFRP2 monoclonal antibody (hSFRP2 mAb) would enhance the activity of a PD-1 inhibitor.

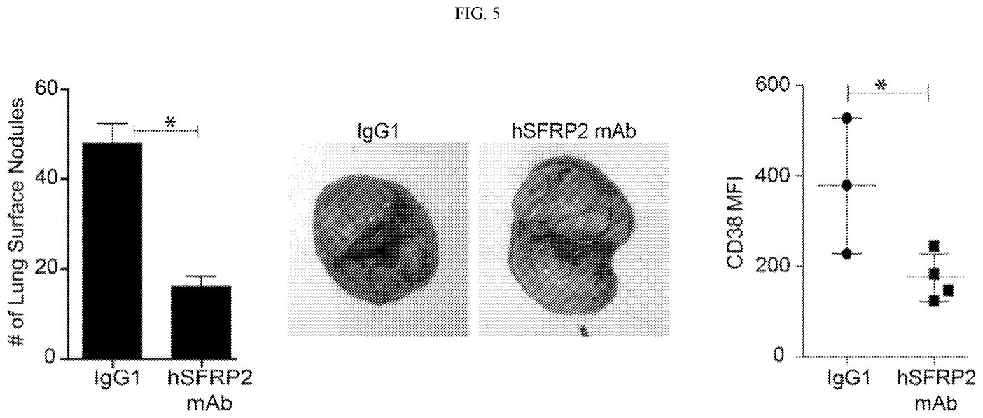

Humanized SFRP2 mAb inhibits metastases in vivo. To evaluate the anti-tumor activity of hSFRP2 mAb in an immunocompetent mouse, the hSFRP2 mAb was tested in a model of tumor metastases, the RF420 murine osteosarcoma, in C57BL/6 mice. RF420 cells were injected in the tail vein of C57BL/6 mice. The presence of metastases in the lungs was verified 7 days after the initial injection of tumor cells. In a first experiment, treatment with hSFRP2 mAb (4 mg/kg injected i.v. every 3 days) started on day 10 after tumor injection and was compared to treatment with IGgl control. At the end point, there was a significant decrease in the number of lung surface nodules with hSFRP2 mAb treatment, compared to control (n=7, p<0.01, FIG. 5). Upon evaluation of cell surface markers for exhaustion we noticed that CD38, which has been shown to tightly be co-expressed with PD-1 (41), was significantly reduced in splenocytes (n=4, p<0.01) from mice treated with hSFRP2 mikb, as compared to the ones treated with IgG1 control (FIG. 5).

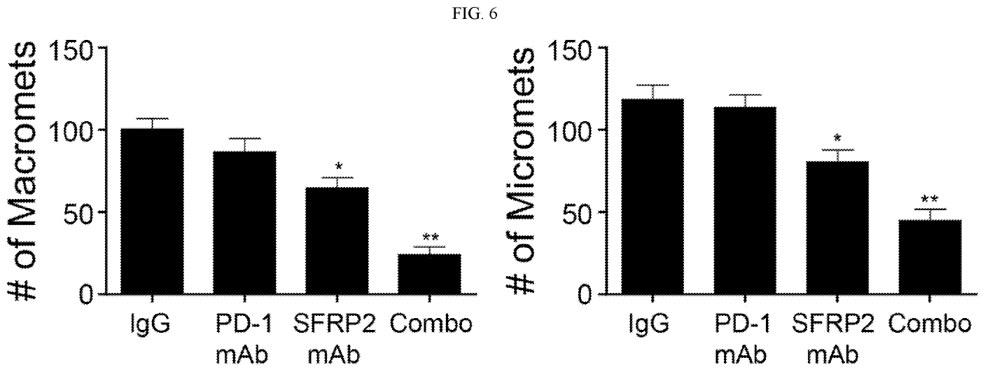

Administration of hSFRP2 mAb with mouse PD-1 inhibitor is effective in inhibiting metastatic osteosarcoma growth in vivo. RF420 mouse osteosarcoma cells were injected in tail vein of C57BL/6 mice. After 7 days, mice were treated with either IgG1 control 4 mg/kg iv weekly, hSFRP2 mAb 4 mg/kg iv every 3 days, mouse PD-1 mAb (200 ug/mouse) every 3 days, or the combination of both antibodies. After 21 days of treatment mice were euthanized and lungs were harvested. The number of surface metastases were counted in each group. The combination of hSFRP2 mAb reduced the number of surface nodules compared to IgG1 control by 75% (FIG. 6).

### Methods of Examples 1-2

Antibodies and Proteins. A control IgG1, omalizumab, was purchased from Novartis (Basel, Switzerland). Human SFRP2 recombinant protein (SFRP2) was prepared as previously described (23) and provided by the Protein Expression and Purification Core Lab at University of North Carolina at Chapel Hill. Humanized SFRP2 monoclonal antibody (hSFRP2 mAb) was produced as previously described and as described in Example 4, and purified of endotoxin.

The following primary antibodies were used in western blots: rabbit anti-CD38 (#146375) and rabbit anti-histone H3 antibodies (#2650s) were from Cell Signaling (Danvers, Mass., USA), rabbit anti-FZDS (#H00007855-D01P, Abnova, Taipei city, Taiwan), mouse anti-PD1 (#66220-1, Proteintech, Rosemont, Ill., USA), rabbit anti-NFATc3 (#SAB2101578) and rabbit anti-actin (#A2103, Sigma-Aldrich, St Louis, Mo., USA). Secondary antibodies were: horse-radish peroxidase (HRP)-conjugated anti-mouse (#7076, Cell Signaling); HRP-conjugated anti-rabbit (#403005, Southern Biotech, Birmingham, Ala., USA). For FACS analysis, rat anti-CD38-PE antibody (#102707) was from BioLegend (San Diego, Calif., USA). Anti-mouse CD3 (#BE00011) and anti-mouse CD28 (#BE0015-1) were from BioXCell (West Lebanon, N.H., USA). The following antibodies were purchased from Biolegend, San Diego, Calif., and used for flow cytometry: anti-CD103 (clone 2E7 cat # 121435), anti-CD5: The gp100 antigen fragment was from AnaSpec (#AS-62589).

Cell Culture. RF420 and mouse osteosarcoma cells, established from a genetically engineered osteosarcoma mouse model (32), were obtained. Cells were cultured at 37° C. in a humidified 5% CO2-95% room air atmosphere. Cell lines were authenticated by ATCC®, and mouse cells tested by Charles River Research Animal (Wilmington, Mass., USA) for rodent pathogens, including mycoplasma whenever they were used in vivo.

Fluorescence Activated Cell Sorting (FACS) analysis of cell proliferation by measure of CarboxyFluorescein Succinimidyl Ester (CFSE) signal intensity. The dilution of CF SE signal tightly correlates with an increase in cell proliferation. Splenic T-cells were pre-labeled with CFSE dye following the instructions of the CellTrace™ CFSE Cell Proliferation Kit (Thermo Fisher Scientific, Waltham, Mass., USA). Cell were then left untreated or activated with soluble anti-CD3 (#BE0001-1, BioXCell, West Lebanon, N.H., USA; 24 ml)/anti-CD28 antibody (#BE0015-1, BioXCell; 2 μg/ml), either alone, or in presence of tumor cells (RF420 or Hs578T breast carcinoma-sarcoma) at 2:1 ratio for 3 days. In addition, some co-cultures were treated with a control IgG1 (10 or with hSFRP2 mAb (10 After 3 days, T-cells from the co-cultures were used to measure CF SE intensity. Mean fluorescence intensity (MFI) was measured by FACS, and analysis was done using FlowJo software.

Western blots. Splenic T-cells were treated for 1 hour with or without SFRP2 (30 nM) or hSFRP2 mAb (10 μM). Control cells for SFRP2 received media alone, and for hSFRP2 mAb experiments received IgG1 10 μM. Cells were then centrifuged at 1000 rpm for 10 min. Medium was removed and cells were stored frozen at −80° C. before being processed. Nuclear extracts were prepared using NE-PER nuclear and cytoplasmic extraction reagent as described in the manufacturer's manual (Pierce Biotechnology, Rockford, Ill.). Splenic T-cells obtained from transgenic Pmel l mice (The Jackson laboratory, Bar Harbor, Me., USA) were treated for 1 hour with or without rhSFRP2 (30 nM) or hSFRP2 mAb (10 μM). Control cells for rhSFRP2 received media alone, and for hSFRP2 mAb experiments received IgG1 10 μM. Cells were then centrifuged at 1000 rpm for 10 min. Medium was removed and cells were stored frozen at −80° C. before being processed. Nuclear extracts were prepared using NE-PER nuclear and cytoplasmic extraction reagent as described in the manufacturer's manual (Pierce Biotechnology, Rockford, Ill.). Protein concentration was measured using Bio-Rad Protein Assay (Bio-Rad Laboratories, Hercules, Calif., USA). Equal amounts of protein were loaded onto SDS-PAGE gels. Proteins were transferred to polyvinylidene difluoride membrane, and western blotting was carried out using the following primary antibodies: rabbit anti-CD38 and rabbit anti-Histone H3 antibodies, rabbit anti-FZD5, mouse anti-PD1, rabbit anti-NFATc3 and rabbit anti-actin. The following secondary antibodies were used: HRP-conjugated anti-mouse, and HRP-conjugated anti-rabbit. The ECL Advance substrate was used for visualization (GE Healthcare Bio-Sciences, Piscataway, N.J., USA).

Next, we evaluated whether hSFRP2 mAb treatment in vitro inhibits CD38 and restores NAD+levels in TGFβ-exposed T-cells in. Spleens were acquired from C57/BL6 mice and a single cell suspension was created and resuspended in ACK lysing buffer for 1 minute with PBS. 1% FCS added to stop the reaction. Once in single cell suspension, CD4⁺and CD8⁺cells were isolated by negative subtraction by using a mix of following antibodies: TCR119, CD25, GR1, NK1.1, CD11C, CD11B, CD19 and incubated on ice for 15 minutes. Cells were incubated with 200 of streptavidin bound beads solution. Following isolation, cells were counted, and 400,000 cells were plated in anti-CD3 (2 μg/mL) and anti-CD28 (5 μg/mL) pre-coated plates. The negative control contained only isolated cells in IL-2 enriched media and no anti-CD3/Cd28 (TCR) coating. Each experimental well with cells contained TCR and IL-2 ((6,000 U/mL) and one of the following experimental conditions: hSFRP2 mAb (10 uM) with or without TGFβ (5 ng/ml). All conditions were done in triplicate and cultured for three days. Following the experiment cells were counted and either stained for FACS or processed for NAD analysis with an NAD/NADH cell-based assay kit. For NAD analysis, at least 250,000 cells were required and processed immediately following the NAD protocol. Cells were centrifuged and incubated under agitation with a permeabilization buffer. After an additional centrifugation, samples and standards were incubated with a reaction buffer for 1h 30 min under agitation. Optical Densities were finally read at 450 nm using a plate reader. For FACs analysis 300,000 cells were resuspended in PBS and incubated in a Live dead stain per manufacturer protocol then washed with PBS, spun down with the supernatant removed. Cells were then suspended in a master mix of antibody and staining buffer (50 μL/sample) containing anti-CD38 PE/Cy5 (1/200), anti-CD4 FITC (1/100), anti-CD8 APC (1/200), anti-PD1 PE (1/200), buffer for 20 min at room temperature and protected from light. The cells were finally fixed in 4% paraformaldehyde for 10-15 minutes before being resuspended in 250 μl of staining buffer.

Metastatic osteosarcoma growth in vivo. In a first experiment, RF420 osteosarcoma cells (5×10⁵) suspended in sterile PBS were injected i.v. via the tail vein of 6-8 weeks old C57BL/6 mice (10 females and 13 males). At day 7, 2 mice were sacrificed, their lungs were removed, fixed in 10% formalin, embedded in paraffin, and stained with hematoxylin and eosin. Sections were screened under the microscope for the presence of metastases. Once the presence of metastases was confirmed, at day 10, mice were treated with IgG1 control 4 mg/kg or hSFRP2 mAb 4 mg/kg (n=10). After 3 weeks of treatment, animals were sacrificed and lungs and spleens were removed. Lung surface nodules were then counted and compared between treatment groups. Spleens were collected fresh for T-cell isolation for flow cytometry.

Next, RF420 cells (5×10⁵) re-suspended in sterile PBS were injected i.v. in the tail vein of 6-8 weeks old C57B1/6 male and female mice purchased from Envigo (Indianapolis, Ind., USA) (strain code 044). Mice were randomly distributed in 4 groups: control (omalizumab, n=13); hSFRP2 mAb (n=11); mouse PD-1 MAb (n=12); PD-1 m Ab+hSFRP2 mAb (n=12). Treatment started 10 days after tumor cell inoculation. Dosage, delivery route and frequency were the following: control (omalizumab) 4 mg/kg i.v. once weekly; hSFRP2 mAb 4 mg/kg i.v. every 3 days; pd-1 mab 8 mg/kg intraperitonealy (i.p.) every 3 days. After 23 days of treatment, animals were sacrificed and their lungs were resected and surface nodules were counted. Surface nodules were counted from pictures of full lungs taken immediately after resection. Lungs were fixed in formalin and embedded in paraffin. They were sectioned and stained with H&E.

Flow cytometry. Staining for CD38, was performed by incubating splenocytes from the experiment with RF420 osteosarcoma injections in the tail vein with primary antibodies to CD38 in FACS buffer (0.1% Bovine Serum Albumin (BSA) in PBS) for 30 min at 4° C. Samples were screen for mean fluorescence intensity (MFI) levels on LSRFortessa, and analyzed with FlowJo software (Tree Star, Oreg.).

Statistics. All power and sample size calculations were performed using PASS version 08.0.13. In vitro experiments were performed in triplicate and repeated three times. Quantitative measures were collected with technician blinded to experimental condition to mitigate potential bias. Group comparisons of continuous measures was performed using two-sample t-tests or ANOVA for two- or multi-group comparisons, respectively.

### Example 3

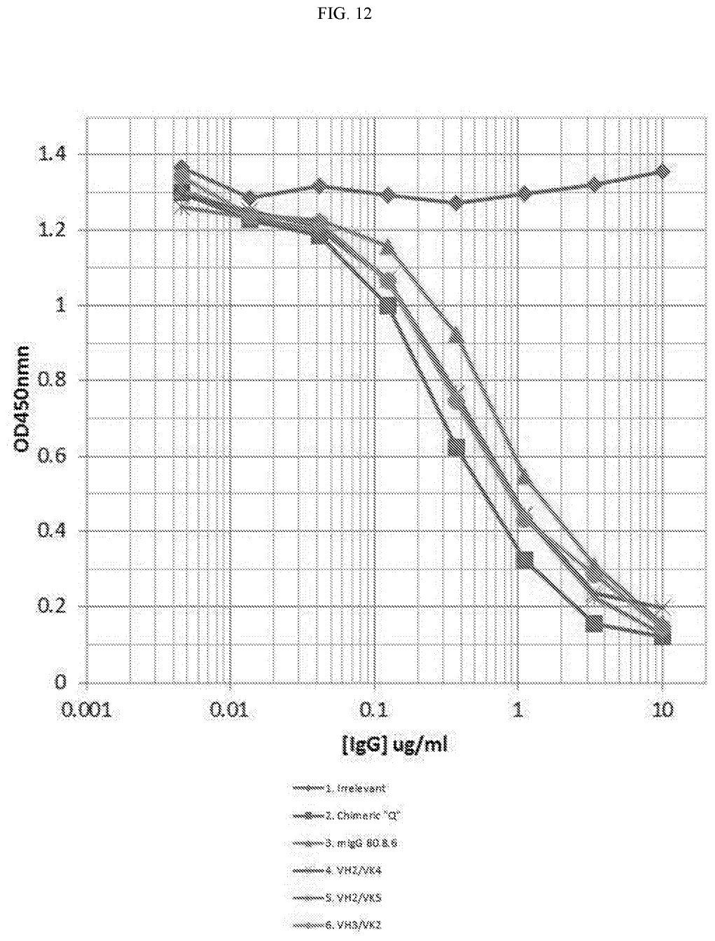

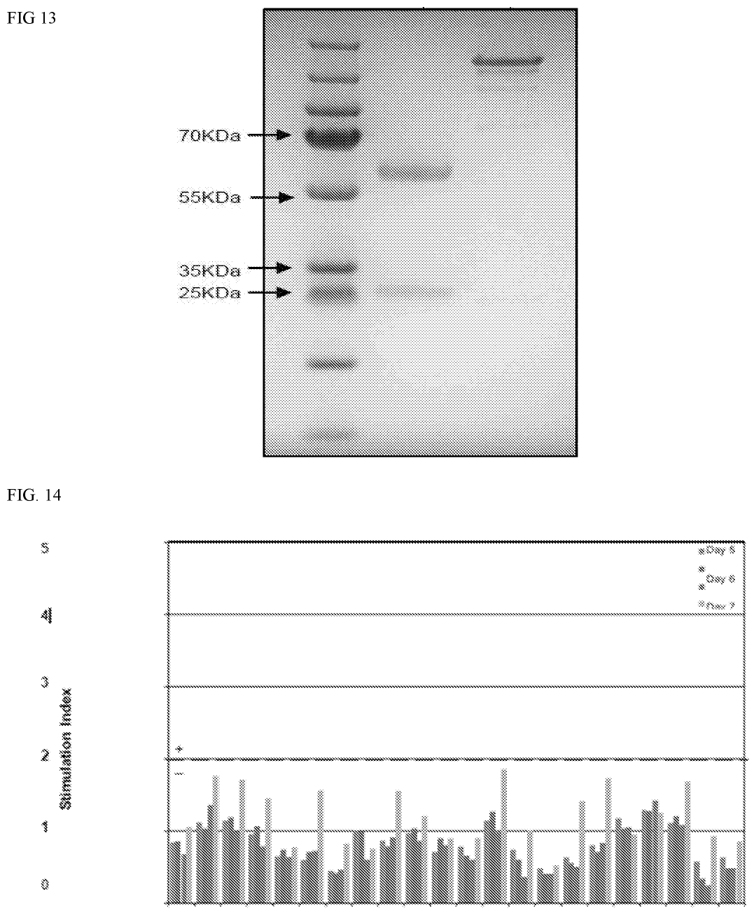

Humanization of SFRP2 mAb. Chimeric antibodies and combinations of composite heavy and light chains (16 antibodies in total) were tested for binding to SFRP2 in a competition ELISA assay. Specifically, a dilution series of purified fully human composite IgG variants were competed against a fixed concentration of biotinylated mouse antibody for binding to SFRP2 Peptide B. Next, bound biotinylated mAb 80.8.6 (mouse SFRP2 mAb) was detected using streptavidin HRP and TMB substrate. This demonstrated that the binding efficiency of all composite antibodies to SFRP2 were broadly comparable to that of the chimeric antibodies, with all variants showing improvement when compared to the murine antibody (FIG. 12). The chimeric antibodies and composite variants of anti-SFRP2 were purified from cell culture supernatants on a Protein A sepharose column, buffer exchanged into PBS pH 7.4 and quantified by OD 280 nm using an extinction coefficient (Ec (0.1%)=1.76) based on the predicted amino acid sequence. Endotoxin testing of the lead hSFRP2 mAb showed endotoxin<0.5EU/m. SDS-PAGE of the lead hSFRP2 mAb showed two bands corresponding to heavy and light chains (FIG. 13). In FIG. 13, SDS Page. 1 μg of purified lead hSFRP2 mAb was loaded on a 4-12% NuPAGE-SDS gel. PageRuler Plus prestained ladder was loaded to allow sizing of bands. Lane 1 was reduced with β-mercaptoethanol; two bands were present for the sample corresponding to the heavy chain and light chain. Lane 2 was non-reduced.

Immunogenicity testing of hSFRP2 mAb. The lead fully humanized and chimeric anti-SFRP2 antibodies were tested against a cohort of 22 healthy donors using EpiScreen™ time course T-cell assay in order to determine the relative risk of immunogenicity. Fully humanized anti-SFRP2 antibody induced no positive responses using SI>2.0, p<0.05 threshold in any of the donors in the proliferation assay, whereas the chimeric anti-SFRP2 antibody induced positive T-cell proliferation responses in 23% of the donors. Results with the control antigen KLH show that there was a good correlation (<10% inter-assay variability) between positive and negative results in repeat studies, which indicates a high level of reproducibility in the assay (FIG. 14). In

FIG. 14, PBMC from bulk cultures were sampled and assessed for proliferation on days 5, 6, 7 and 8 after incubation with the three test samples. Proliferation responses with an SI>2.0 (p<0.05), indicated by dotted line that were significant (p<0.05) using an unpaired, two sample student's t test were considered positive in this Figure.

Humanized SFRP2 mAb binds SFRP2 with high affinity. To determine binding affinity of the lead hSFRP2 mAb to SFRP2, rhSFRP2 (1 μM) was incubated with increasing concentrations of hSFRP2 mAb in a microplate solid phase protein binding ELISA assay. hSFRP2 mAb bound rhSFRP2 with an EC50 of 8.72 nM and a Kd of 74.1 nM. FIG. 1A shows that humanized SFRP2 mAb binds rhSFRP2 with high affinity and produced a concentration-response curve showing the 480 nm absorbance measured after binding increasing concentrations of hSFRP2 mAb to a preset concentration of 1 μM rhSRP2 in an ELISA assay (n=16).

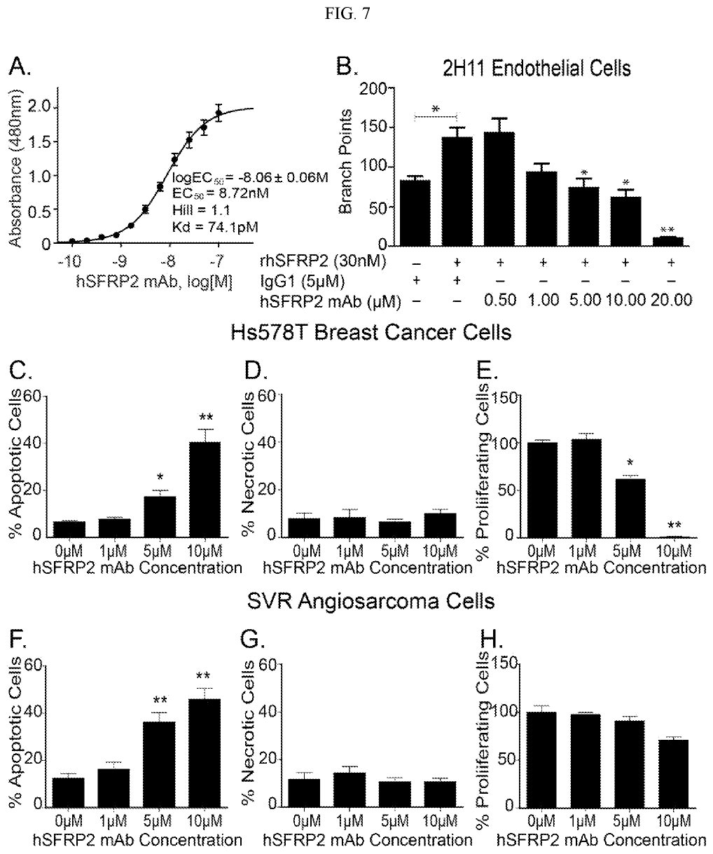

Humanized SFRP2 mAb inhibits endothelial tube formation, tumor cell proliferation, and promotes tumor apoptosis. Consistent with previous reports; rhSFRP2 induced an increase in the number of branch points, compared to control cells (n=4, p<0.05). FIG. 7B is a bar graph showing the effects of rhSFRP2 and hSFRP2 mAb on 2H11 endothelial tube formation. To obtain this data, 2H11 cells were incubated and either treated with IgG1 control only (5 or IgG1 (5+rhSFRP2 protein (30 nM), or a combination of rhSFRP2 (30 nM) and hSFRP2 mAb (from 0.5 to 10 n=4*: p<0.05; **: p<0.001. Conversely, increasing concentrations of hSFRP2 mAb significantly counteracted rhSFRP2 effects on tube formation (n=4, p<0.05). The IC₅₀ for hSFRP2 mAb inhibition of SFRP2-stimulated tube formation was 4.9±2 μM.

The effects of rhSFRP2 mAb on tumor cell proliferation, apoptosis and necrosis in Hs578T human carcinoma/sarcoma breast cancer and mouse SVR angiosarcoma cells was evaluated in vitro. Treatment with hSFRP2 mAb increased tumor apoptosis significantly in both Hs578T breast cancer (FIG. 7C; p<0.05 and p<0.001 for 5 μM and 10 μM hSFRP2 mAb, respectively) and SVR angiosarcoma cells (FIG. 7F; p<0.001 for both 5 μM and 10 hSFRP2 mAb), with no change in necrosis. Treatment with hSFRP2 mAb had no effect on SVR proliferation (FIG. 7H), but significantly reduced tumor cell proliferation of Hs578T breast cancer cells (FIG. 7E, 5 μM p<0.05, 10 μM p<0.001).

Determination of efficacy and toxicity hSFRP2 mAb in vivo. Mice inoculated with SVR angiosarcoma cells were treated with hSFRP2 mAb doses at 2, 4, 10 and 20 mg/kg i.v. every three days; or IgG1 control, for 21 days. There was no weight loss or lethargy in any of the antibody treated mice. There were no pathologic changes in the liver or lungs, even at the 20 mg/kg dose. At the end of the experiment the weights remained similar among the groups (32.2±1.4 g for controls; 31.3±1.1 g for 2 mg/kg; 32.1±0.5 g for 4 mg/kg; 31.8±0.9 g for 10 mg/kg and 32.7±1.0 g for 20 mg/kg. The dose with the maximum effect was 4 mg/kg, where there was a 69% reduction in tumor volume (n=5 per group, p=0.05).

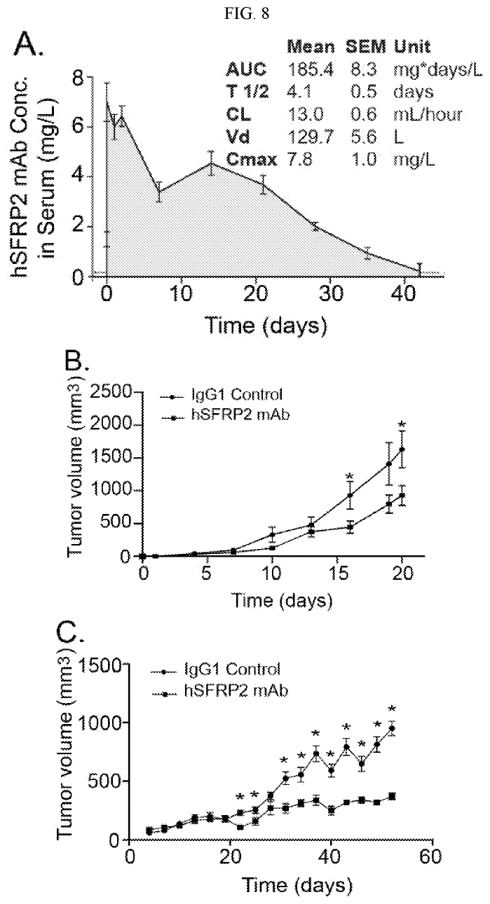

To study the pharmacokinetic properties of the antibody, a single dose of hSFRP2 mAb 4 mg/kg via the tail vein was injected in to nude mice and blood samples were collected at different time points (FIG. 8). Recombinant hSFRP2 treatment led to increased membrane CD38 and nuclear NFATc3 protein, while hSFRP2 mAb inhibits the accumulation of nuclear NFATc3 in T-cells. The data in FIG. 8A demonstrates that the FZD5 protein is present in T-cells. FIG. 8A is a pharmacokinetic plot showing the decrease in concentration of hSFRP2 mAb in the serum of mice over time after a single i.v. injection of 4 mg/kg. The half-life of the antibody in the serum of the animals was 4.1±0.5 days with a maximum serum concentration (Cmax) of 7.8±1.0 mg/L and a clearance (CL) of 13.0±0.6 mL/hour.

To confirm the efficacy of the dose identified in the MTD experiment, the inventors repeated the experiment with the SVR angiosarcoma tumors on a larger number of animals (n=10 animals/group) and started treating them with 4 mg/kg hSFRP2 mAb. T-cells were treated with rhSFRP2 (30 nM) for 1h, and processed using the NE-PER kit to separate cytoplasmic and nuclear fractions (FIG. 8B). Samples were probed with antibodies to the indicated protein markers, and levels of proteins in treated cells were compared to those in untreated cells. After 3 weeks, tumors treated with hSFRP2 mAb were 43% smaller than tumors treated with the IgG1 control (1,631.3±283 mm³ for control, 928.5±148 mm³ for hSFRP2 mAb; p<0.05).

Next, the inventors considered whether hSFRP2 mAb could affect the growth of other tumor types. Mice with Hs578T breast carcinoma-sarcoma xenografts were treated with hSFRP2 mAb or IGg1 control. T-cells were treated with antigen gp100 (0.87 μM) or hSFRP2 mAb (10 μM) alone or in combination for 60 min and nuclear fractions isolated (FIG. 8C). Protein levels of NFATc3 in rhSFRP2-treated cells were compared to those in untreated cells. Comparison between control and each treated group at each time point showed that treatment days 22, 25, and all time points from day 31 from baseline are statistically significant (p=0.05). In fact, there was a 61% reduction in tumor volume in the hSFRP2 mAb treated mice, n=11, *P<0.05). Additionally, there was no weight loss or lethargy in any of the treated mice.

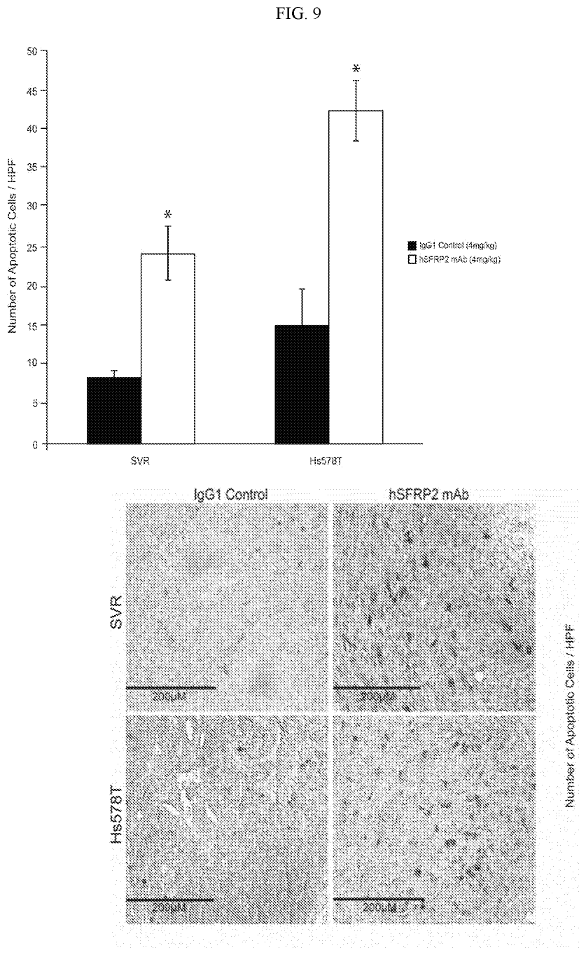

Humanized SFRP2 mAb induces apoptosis in tumors in vivo. hSFRP2 mAb induces apoptosis in vitro, and inhibits proliferation in breast cancer cells and the inventors investigated if these phenotypes were retained in vivo. While the proportion of proliferative (Ki67-positive) cells was not affected by hSFRP2 mAb treatment, compared to IgG1 control tumors (23±1.6% vs. 29±4.2% for SVR tumors; 18±2.7% vs. 18±2.8% for Hs578T tumors, p=NS), the proportion of apoptotic cells increased by 188% in SVR tumors (8.4±0.9 in IgG1 control, 24.2±3.5 in hSFRP2 mAb tumors; n=10, p<0.05) and by 181% in Hs578T tumors (15.1±4.9 in IgG1 control, 42.4±3.9 in hSFRP2 mAb tumors; n=10, p<0.05)(FIG. 9).

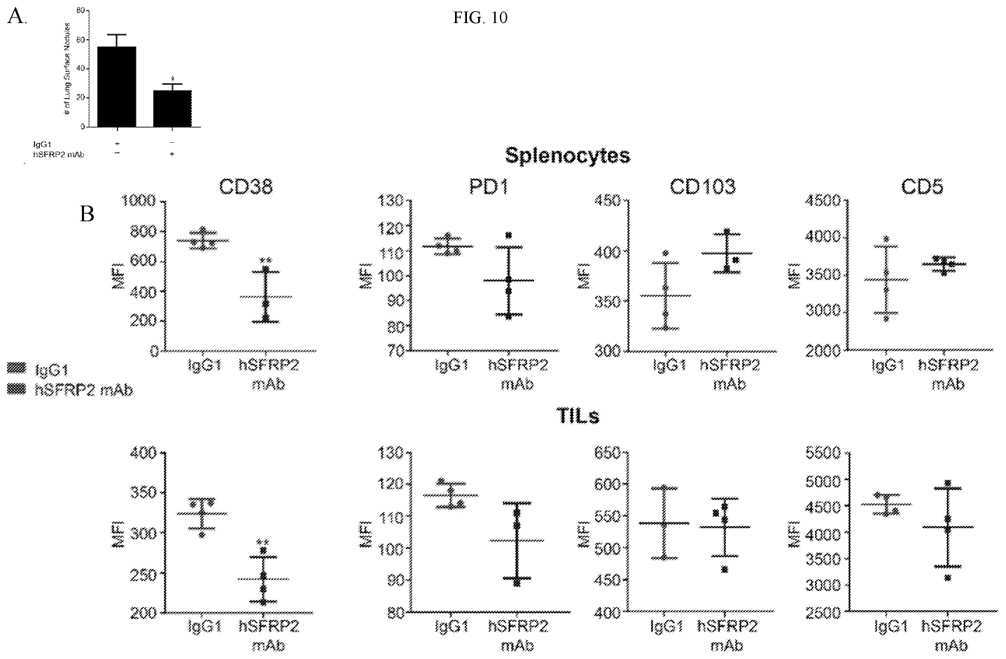

To evaluate the anti-tumor activity of hSFRP2 mAb in an immunocompetent mouse, the inventors tested the hSFRP2 mAb in RF420 murine osteosarcoma in C57BL/6 mice in a model of tumor metastases. RF420 osteosarcoma cells were injected in the tail vein of C57BL/6 mice. On day 10 treatment with IgG1 control or hSFRP2 mAb was begun. Mice were euthanized on day 21 of treatment and surface nodules were counted. There was a significant reduction in the number of surface nodules in mice treated with hSFRP2 mAb compared to control (n=7, p<0.01, FIG. 10A). Upon evaluation of cell surface markers for exhaustion the inventors noticed that CD38, which has been shown to tightly co-express with PD-1, was significantly reduced on the splenocytes (n=4, p<0.01) and TILS (n=4, p<0.01) in mice treated with hSFRP2 mAb as compared to the ones from IgG control, with no significant difference in PD-1, CD103, and CD5 (n=3)(FIG. 6B). The expression of other exhaustion marker such as PD-1, CD103, TNFα, or CD5 were not significant in splenocytes or TiLs (n=4, p=NS).

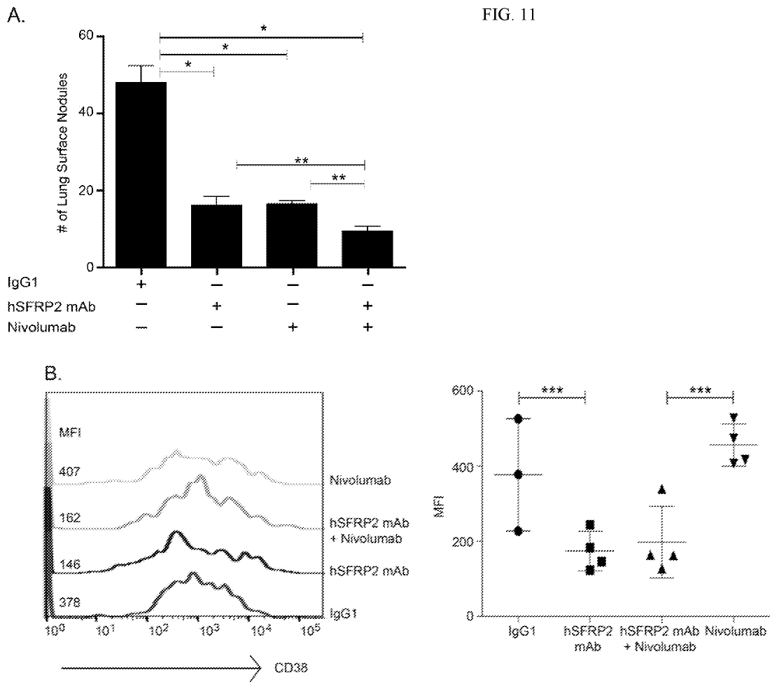

In a second osteosarcoma experiment, osteosarcoma RF420 cells were injected intravenously in immunocompetent mice. The study was divided into four groups. The first group was treated with hSFRP2 mAb 4 mg/kg i.v. every 3 days. There was also a IGg1 control group, a group who was administered nivolumab, an anti-PD-1 antibody, every 3 days at 8 mg/kg i.v., and a group who received both hSFRP2 mAb and the anti-PD-1 antibody. The treatment started on day 10 after injection and three weeks later, the animals were euthanized, their lungs were resected and surface nodules were counted *: p<0.0001; **: p<0.01, n=12). These groups were compared to measure the development of lung metastasis. Each individual treatment reduced the number of surface nodules compared to IgG1 control (43.6±6.8 for IgG1 control, 18.3±3.4 for hSFRP2 mAb, 16.3±1.1 for nivolumab; p<0.0001, FIG. 11A). There was an 80% reduction in the incidence of metastatic lesions comparing mice treated with the combination of hSFRP2 mAb and nivolumab to mice treated with IgG1 control (IRR=0.20, 95% CI=0.13 to 0.32; p<0.0001). There is a 51% reduction in the incidence of metastatic lesions comparing mice treated with the combination of hSFRP2 mAb and nivolumab and to mice treated with single agent hSFRP2 mAb (IRR=0.49, 95% CI=0.31 to 0.77; p=0.0021). There is a 45% reduction in the incidence of metastatic lesions comparing mice treated with the combination of hSFRP2 mAb and nivolumab and to mice treated with single agent nivolumab (IRR=0.55, 95% CI=0.35 to 0.86; p=0.0084) (FIG. 11A).

The inventors measured the impact of nivolumab and hSFRP2 mAb, given as individual treatments or in combination, on CD38 levels in mouse T-cells. Specifically, T-cells were isolated from spleens of C57BL/6 mice injected with RF420 cells and treated with IgG1 control, hSFRP2 mAb, nivolumab, or a combination of hSFRP2 mAb and nivolumab. Cells were then stained with a CD38 labeled with a fluorochrome and mean fluorescent intensity (MFI) was analyzed by FACS. Nivolumab treatment alone had no effect on CD38 levels. However, hSFRP2 mAb reduced CD38 surface expression in T-cells as compared to the T-cells that were obtained from the group treated with control IgG antibody (p<0.001, FIG. 11B), indicating that targeting SFRP2 is sufficient to reduce the expression of CD38 on T-cells. These results support the proposition that hSFPR2 mAb administration could restore the T cell immune response and prevent tumor growth. It should be noted that because nivolumab is a human antibody, it is not best suited for treatment in a murine model.

Humanization of SFRP2 monoclonal antibody. V region genes encoding the murine SFRP2 monoclonal antibody 80.8.6 (21) were initially cloned, and used to construct chimeric antibodies comprising the murine V regions combined with human IgG1 heavy chain constant regions, and κ light chain constant regions. The chimeric antibodies and combinations of composite heavy and light chains (16 antibodies in total) were expressed in NSO or HEK293 cells, purified and tested for binding to SFRP2 peptide in a competition ELISA assay.

Immunogenicity testing. The lead fully-humanized anti-SFRP2 antibody (VH2/VK5) and the reference chimeric anti-SFRP2 antibody were assessed for immunogenic potential using EpiScreen™ time course T-cell assays, where bulk cultures were established using CD8⁺depleted PBMCs, and T-cell proliferation was measured at various time points by incorporation of [³H]-Thymidine after the addition of the samples. The lead fully humanized and chimeric anti-SFRP2 antibodies were tested against a cohort of 22 healthy donors using EpiScreen™ time course T-cell assay in order to determine the relative risk of non-specific immunogenicity. The samples were tested at a final concentration of 50 μg/ml based on Antitope's previous studies showing that this saturating concentration is sufficient to stimulate detectable antibody-specific T-cell responses. In order to assess the immunogenic potential of each sample, the EpiScreen™ time course T-cell assay was used with analysis of proliferation to measure T-cell activation. Since the samples had not been previously assessed in a PBMC-based assay, an initial assessment of any gross toxic effect of the samples on PBMC viability was determined. Cell viabilities were calculated using trypan blue dye exclusion of PBMC, 7 days after culture with the test samples.

Antibodies and Proteins. The following primary antibodies were used in western blots: rabbit anti-CD38 (#14637s) and rabbit anti-histone H3 antibodies (#2650s) were from Cell Signaling (Danvers, Mass., USA), rabbit anti-FZDS (#H00007855-D01P, Abnova, Taipei city, Taiwan), mouse anti-PD1 (#66220-1, Proteintech, Rosemont, Ill., USA), rabbit anti-NFATc3 (#SAB2101578) and rabbit anti-actin (#A2103, Sigma-Aldrich, St Louis, Mo., USA). Secondary antibodies were: HRP-conjugated anti-mouse (#7076, Cell Signaling); HRP-conjugated anti-rabbit (#403005, Southern Biotech, Birmingham, Ala., USA). For ELISA, HRP conjugated goat anti-human IgG from Abcam, Cambridge, Mass., USA. For FACS analysis, rat anti-CD38-PE antibody (#102707) was from BioLegend (San Diego, Calif., USA). Anti-mouse CD3 (#BE00011) and anti-mouse CD28 (#BE0015-1) were from BioXCell (West Lebanon, N.H., USA). A control IgG1, omalizumab, was purchased from Novartis (Basel, Switzerland). Human SFRP2 protein (rhSFRP2) was prepared as previously described. The gp100 antigen fragment was from AnaSpec (#AS-62589).

Microplate Solid Phase Protein Binding (ELISA) Assay to Determine Binding Affinity of rhSFRP2 to hSFRP2 mAb. A microplate solid phase protein binding assay was used to determine the EC₅₀ for rhSFRP2 and hSFRP2 mAb. Flat-bottom Ni²⁺coated 96-well microplates (#15442, Thermo Fisher Scientific, Waltham, Mass., USA) were blocked with 0.05% bovine serum albumen (BSA, #001-000-162, Jackson ImmunoResearch, West Grove, Pa., USA) in phosphate buffered saline (PBS, #BP399-1, Fisher Scientific, Waltham, Mass., USA) overnight at 4° C. 1 μM his-tagged rhSFRP2 diluted in PBS (pH 7.4) was incubated on the blocked plate overnight at 37° C. The plates were washed 3 times with 250 μl/well of PBS. Increasing doses of hSFRP2 mAb in PBS (0 pM, 100 pM, 200 pM, 400 pM, 800 pM, 1.6 nM, 3.15 nM, 6.3 nM, 12.5 nM, 25 nM, 50 nM, 100 nM) were incubated on the plate with rhSFRP2 at 37° C. overnight. Plates were washed 3 times, blocked for 1 hour at room temp in 0.1% BSA in PBS, and subsequently incubated with 100 μl/well of secondary antibody (HRP conjugated goat anti-human IgG), diluted 1:40,000 in PBS, for 1 hour at 37° C. After plates were washed 5 times, each well was incubated with 100 μl K-Blue TMB substrate (#308176, Neogen, Lexington, Ky., USA) for 5 minutes in the dark. The reaction was stopped with 100 ul 2N H2504. Absorbance was read at 450 nm. EC₅₀ calculations were determined via non-linear regression analysis with variable slope using GraphPad Prism log (inhibitor) vs. normalized response—variable slope function with top constrained to 100%. EC₅₀ was converted to Kd using the Cheng-Prusoff equation where agonist concentration and EC₅₀ were equal(40). Results are expressed as means±standard error of the mean. Each data point is the result of 8 independent measurements (n=8).

Cell Culture. 2H11 mouse endothelial cells (#CRL-2163, ATCC®, Manassas, Va., USA) were cultured in Opti-MEM (#22600134, Thermo Fisher Scientific, Waltham, Mass., USA) with 5% heat inactivated fetal bovine serum (FBS, #FB-12, Omega Scientific, Biel/Bienne, Switzerland) and 1% penicillin/streptomycin (v/v). Hs578T human breast carcinoma-sarcoma triple negative cells (#30-202, ATCC®, Manassas, Va., USA) were cultured in DMEM (ATCC®) with 10% FBS, 0.01 mg/ml bovine insulin (#I0516, Sigma-Aldrich, St. Louis, Mo., USA) and 1% penicillin/streptomycin (#MT30009C, Thermo Fisher Scientific). SVR angiosarcoma cells were obtained from American Type Culture Collection (#CRL-2280, ATCC®) and cultured in Opti-MEM (Thermo Fisher Scientific) with 8% FBS and 1% penicillin/streptomycin (v/v). RF420 mouse osteosarcoma cells, established from a genetically engineered osteosarcoma mouse model (41), were obtained from Dr. Jason T. Yustein (Texas Children's Cancer and Hematology Centers, Department of Pediatrics, Baylor College of Medicine, Houston, Tex., USA) and were cultured in DMEM (ATCC®) with 10% heat-inactivated FBS and 1% penicillin/streptomycin (v/v). All cell lines were cultured at 37° C. in a humidified 5% CO2-95% room air atmosphere. All cell lines were authenticated by ATCC®, and mouse cells tested by Charles River Research Animal (Wilmington, Mass., USA) for rodent pathogens, including mycoplasma whenever they were used in vivo. Murine T-cells were isolated from C57BL/6 mice and gp100 reactive TCR bearing Pmel transgenic mice on C57BL/6 background obtained from Jackson Laboratory (Bar Harbor, Me., USA).

Endothelial tube formation assay. 2H11 endothelial cells were plated in Opti-MEM with 5% FBS and allowed to settle for 24 hrs. Quiescence was induced by maintaining the cells in Opti-MEM with 2.5% FBS overnight. Matrigel™ (#ECM625, Millipore, Bedford, Mass., USA), was polymerized in the wells of a 96 well plate according to the In Vitro Angiogenesis Assay protocol. In this assay, nine treatment conditions were prepared: IgG1 alone (5 μM; omalizumab); rhSFRP2 protein (30 nM) with IgG1 (5 μM); or rhSFRP2 (30 nM) combined with increasing concentrations of hSFRP2 mAb (0.5, 1, 5, 10 or 20 μM). Treatments resuspended in Opti-MEM with 2.5% FBS were pre-incubated on a rocker at 37° C., 5% CO2, for 90 minutes prior to adding them to the cells. 1.9×10⁴ cells were resuspended in 150 μl of pre-incubated treatments, then incubated for an additional 30 min on a rocker at 37° C., 5% CO2. Finally, the cell suspension was added to each well already coated with polymerized Matrigel™. Each experiment was repeated 4 independent times, with n=4 per condition. Control cells were given fresh Opti-MEM with 2.5% FBS and 5 μM IgG1. For each treatment condition, after 4h of incubation at 37° C., 5% CO2, images were acquired using the 4X objective lens of the EVOS FL Digital Imaging System (Thermo Fisher Scientific, Waltham, Mass., USA). Branch points were counted using ImageJ Angiogenesis Analysis software (National Institutes of Health, Bethesda, Md., USA). In the GraphPad Prism software, data was analyzed to determine IC50 using non-linear regression and the Dose-response—Inhibition family of equations.

Proliferation Assay. Hs578T breast carcinoma-sarcoma and SVR angiosarcoma cells were plated in a 96 well plate at 3,000 cells/well. After 4 hours, hSFRP2 mAb (1, 5, or 10 μM) was added to the growth medium at the indicated concentrations. Cells were allowed to incubate for 72 hours at 37° C., 5% CO2. Proliferation was assessed using the Cyquant Direct Cell Proliferation Assay Kit (#C35011, Thermo Fisher Scientific, Waltham, Mass., USA). Images were acquired using the EVOS FLc Digital Imaging System (Thermo Fisher Scientific). Cells were counted using the FIJI cell counting software.

Apoptosis/Necrosis. Hs578T breast carcinoma-sarcoma breast and SVR angiosarcoma cells were plated in 16 well chamber slides (#178599, Thermo Fisher Scientific, Waltham, Mass., USA) at 2×10⁴, 3×10⁴, and 7.5×10³ cells/well, respectively. The next day, cells were incubated at 37° C., 5% CO2 with 1, 5 or 10 μM of hSFRP2 mAb or 5 μM of IgG1 control in suspension with growth medium for 2 hours. Necrosis and apoptosis were determined following the protocol of the Apoptotic/Necrotic Detection kit (#PK-CA707-30017, PromoCell, GmbH, Heidelberg, Germany). Images were acquired using the EVOS FLc Digital Imaging System (Thermo Fisher Scientific, Waltham, Mass., USA). Cells were counted using ImageJ cell counting software. Each data point was the result of 2 independent experimental repeats, each containing 4 separate wells (total n=8).

Western blots. Splenic T-cells obtained from transgenic Pmell mice (The Jackson laboratory, Bar Harbor, Me., USA) were treated for 1 hour with or without rhSFRP2 (30 nM) or hSFRP2 mAb (10 μM). Control cells for rhSFRP2 received media alone, and for hSFRP2 mAb experiments received IgG1 10 μM. Cells were then centrifuged at 1000 rpm for 10 min. Medium was removed and cells were stored frozen at −80° C. before being processed. Nuclear extracts were prepared using NE-PER nuclear and cytoplasmic extraction reagent as described in the manufacturer's manual (Pierce Biotechnology, Rockford, Ill.). Protein concentration was measured using Bio-Rad Protein Assay (Bio-Rad Laboratories, Hercules, Calif., USA). Equal amounts of protein were loaded onto SDS-PAGE gels. Proteins were transferred to polyvinylidene difluoride membrane, and western blotting was carried out using the following primary antibodies: rabbit anti-CD38 and rabbit anti-Histone H3 antibodies, rabbit anti-FZDS, mouse anti-PD1, rabbit anti-NFATc3 and rabbit anti-actin. The following secondary antibodies were used: HRP-conjugated anti-mouse, and HRP-conjugated anti-rabbit. The ECL Advance substrate was used for visualization (GE Healthcare Bio-Sciences, Piscataway, N.J., USA).

FACS analysis of cell proliferation by measure of CFSE signal intensity. The dilution of CF SE signal tightly correlates with an increase in cell proliferation. Splenic T-cells from Pmel1 transgenic mice were pre-labeled with CF SE dye following the instructions of the CellTrace™ CFSE Cell Proliferation Kit (Thermo Fisher Scientific, Waltham, Mass., USA). Cell were then left untreated or activated with soluble anti-CD3 (#BE0001-1, BioXCell, West Lebanon, N.H., USA; 24 ml)/anti-CD28 antibody (#BE0015-1, BioXCell; 2 μg/ml), either alone, or in presence of tumor cells (SVR angiosarcoma or Hs578T breast carcinoma-sarcoma) at 2:1 ratio for 3 days. In addition, some co-cultures were treated with a control IgG1 (10 μM), or with hSFRP2 mAb (10 μM). After 3 days, T-cells from the co-cultures were used to measure CFSE intensity. Mean fluorescence intensity (MFI) was measured by FACS, and analysis was done using FlowJo software.

Maximum Tolerated Dose (MTD) of hSFRP2 mAb in vivo. Animal experiment protocols were consistent with NIH guidelines for the care and use of laboratory animals. 10⁶ SVR angiosarcoma cells were injected subcutaneously into the right flank of 6 week old nude male and female mice obtained from Charles River (Wilmington, Mass., USA). The following day, mice (n=5 per group) were treated i.v. with PBS control with various concentrations of purified hSFRP2 mAb (2, 4, 10, and 20 mg/kg) injected via the tail vein every 3 days. Animal were treated and tumor volumes were measured every three days until control tumors reached an average diameter of 2 cm, which was defined as the end-point. After euthanasia, tumors, lungs and livers were harvested and fixed in 10% formalin.

Pharmacokinetic study. Male and female C57BL/6 mice were injected with 4 mg/kg of hSFRP2 mAb at different time points (0, 5 min, 1, 2, 7, 14, 21, 28, 35, and 42 days). Three mice were used for each time point (n=3). At the end point, blood samples were taken through the portal vein and placed in separator tubes (#367981, Becton Dickinson, Franklin Lakes, N.J., USA). Samples were centrifuged at 1300×g for 15 min.

Microplate Solid Phase Protein Binding (ELISA) Assay for Pharmacokinetics (PK) of hSFRP2 mAb. Flat-bottom Ni²⁺coated 96-well microplates were blocked with 0.05% BSA in PBS overnight at 4° C. 1 μM his-tagged rhSFRP2 diluted in PBS (pH 7.4) was incubated overnight at 37° C. The plates were washed 3 times with 250 μl/well of PBS. Then, a 1:50 dilution of mouse serum was added to the plate and incubated shaking gently at 37° C. overnight. Plates were washed 3 times, blocked for 1 hour at room temp in 0.1% BSA in PBS, and subsequently incubated with 100 μl/well of secondary antibody (HRP conjugated goat anti-human IgG), diluted 1:40,000 in PBS for 1 hour at 37° C. After plates were washed 5 times, each well was incubated with 100 μl K-Blue TMB substrate for 5 minutes in the dark. The reaction was stopped with 100 μl 2N H2504. Absorbance was read at 450 nm. For PK estimates of AUC, t1/2, CL, Vd, Tmax and Cmax were determined using non-compartmental analysis (NCA) in EXCEL and (42). NCA uses the linear trapezoidal rule to determine the area under the plasma concentration-versus-time curve (AUC). T_{1/2} represents the terminal half-life. For AUC calculations, nM concentrations from ELISA were converted to mg/L.

Angiosarcoma allografts in vivo. 10⁶ SVR angiosarcoma cells were injected subcutaneously into the right flank of 6 week old nude male and female mice obtained from Charles River (Wilmington, Mass., USA). The following day, mice (n=10 animals/group) were injected i.v. with hSFRP2 mAb (4 mg/kg) or IgG1 control (omalizumab 4 mg/kg) via the tail vein, and were treated every 3 days. Serial caliper measurements of perpendicular diameters performed twice a week were used to calculate tumor volume using the following formula: [(L (mm)×W (mm)×H (mm))×0.5]. Mice were monitored daily for body conditioning score and weight. Mice were sacrificed when the controls reached 2 cm diameter, and tumors were resected and fixed in formalin and embedded in paraffin.

Hs578T breast carcinoma-sarcoma xenografts in vivo. Hs578T xenografts were established in 5- to 6-week-old nude female mice from Charles River (Wilmington, Mass., USA). Mice were inoculated in the mammary fat pad with 10⁶ cells and treatment began when the average tumor size was approximately 100 mm³ (day 30). Animals were treated with 4 mg/kg hSFRP2 mAb (n=11) injected i.v. every 3 days or with 4 mg/kg IgG1 control (n=11) until the end-point, when control tumors reached 2 cm in diameter. Tumors were measured twice weekly using a caliper, and tumor volumes were then calculated, as described above. Tumors were resected, fixed in formalin, and embedded in paraffin.

RF420 metastatic osteosarcoma in vivo. In a first experiment, RF420 osteosarcoma cells (5×10⁵) suspended in sterile PBS were injected i.v. via the tail vein of 6-8 weeks old C57BL/6 mice (10 females and 13 males). At day 7, 2 mice were sacrificed, their lungs were removed, fixed in 10% formalin, embedded in paraffin, and stained with hematoxylin and eosin. Sections were screened under the microscope for the presence of metastases. Once the presence of metastases was confirmed (at day 10, mice were treated with IgG1 control 4 mg/kg or hSFRP2 mAb 4 mg/kg (n=10). After 3 weeks of treatment, animals were sacrificed and lungs were removed. Surface nodules were counted. In the second experiment, RF420 cells (5×10⁵) re-suspended in sterile PBS were injected i.v. in the tail vein of 6-8 weeks old C57B1/6 male and female mice purchased from Envigo (Indianapolis, Ind., USA) (strain code 044). Mice were randomly distributed in 4 groups: control (omalizumab, n=13); hSFRP2 mAb (n=11); nivolumab (NDC#0003-3772-11, Bristol-Meyers Squibb, Princeton, N.J.) (n=12); nivolumab+hSFRP2 mAb (n=12). Treatment started 10 days after tumor cell inoculation. Dosage, delivery route and frequency were the following: control (omalizumab) 4 mg/kg i.v. once weekly; hSFRP2 mAb 4 mg/kg i.v. every 3 days; nivolumab 8 mg/kg i.p. every 3 days. After 23 days of treatment, animals were sacrificed and their lungs were resected and surface nodules were counted. Surface nodules were counted from pictures of full lungs taken immediately after resection. Spleens were collected fresh for T-cell isolation, immunohistochemistry and tunnel assay.

Immunohistochemistry. Formalin fixed, paraffin embedded tumor sections were deparaffinized twice for ten minutes in Xylene and hydrated twice in absolute ethanol, twice in 95% ethanol, and then tap water. Slides were incubated in 3% hydrogen peroxide for ten minutes at room temperature followed by two washes in PBS 1 ×. A citrate buffer antigen retrieval step was performed in a vegetable steamer using the kit Vector Antigen Retrieval Citrate Buffer pH6 (H-3300) for 40 minutes with 10 minutes to cool. Slides were incubated in blocking serum provided in the Vector Rabbit IMPRESS HRP Kit (MP-4100) in a humidified slide chamber at room temperature for 1 hour. The blocking serum was then drained off, and the slides were incubated overnight at 4° C. with the Ki67 antibody 1:40 dilution (PA1-21520). The next day, the slides were rinsed 3 times in PBS for 5 min/wash. The secondary antibody from the Vector Rabbit IMPRESS HRP Kit was added and the slides were incubated for 30 min RT, and then rinsed 3 times in PBS for 5 min/wash. DAB solution was prepared and added to the slides as instructed in the Vector DAB kit (SK-4100) for 5 min, rinsed in PBS, and counterstained with hematoxylin for 30 seconds. Slides were then washed in distilled water, followed by ammonia alcohol, dehydrated twice in 95% ethanol, twice in 100% ethanol, twice in xylene, and then mounted with a coverslip. Tumor proliferation was quantified as the number of positively stained cells/unit area, using the average of 3 fields per slice.

TUNEL assay. Sections from Hs578T and SVR tumors were stained for apoptotic cells following the manufacturer protocol for the Apoptag® Peroxidase In Situ Apoptosis Detection Kit (#S7100). All sections were deparaffinized with Histoclear (#HS-200, National Diagnostics, Atlanta, Ga., USA). The following materials were not supplied with the TUNEL kit and were purchased separately: 30% Hydrogen peroxide (#5155-01, J. T. Baker, Phillipsburg, N.J., USA), Proteinase K (#21627, Millipore, Burlington, Mass., USA), Metal enhanced DAB substrate kit (#34065, Thermoscientific, Waltham, Mass., USA), stable peroxidase substrate buffer 1× (#1855910, Thermoscientific, Waltham, Mass., USA) and 1-Butanol (#B7908, Sigma-Aldrich, St. Louis, Mo., USA). Five fields were randomly selected in each sample and photographed using the EVOS FLc microscope (Life Technologies Inc., Waltham, Mass., USA). In each field, tumor apoptosis was quantified as the number of apoptotic nuclei/HPF.

Flow cytometry. Staining for CD38 surface expression was performed by incubating splenocytes from the experiment with RF420 osteosarcoma injections in the tail vein with rat anti-CD38-PE antibody (1:200; #102707, Biolegend, San Diego, Calif., USA) in FACS buffer (0.1% BSA in PBS) for 30 min at 4° C. Samples were screen for CD38 mean fluorescence intensity (MFI) levels on LSRFortessa, and analyzed with FlowJo software (Tree Star, Oreg.).

Statistics. For in vitro assays, statistical differences between IgG1 and hSFRP2 mAb treatments were calculated using a two-tailed student's t-test, with p<0.05 considered significant. For in vivo tumor studies in angiosarcoma (where treatment was started day after tumor inoculation), a two-tailed student's t-test was used. For Hs578T, where treatment was started on day 30 when tumors were palpable, the data was normalized to adjust for differences in baseline tumor volumes, by dividing tumor volume from day 34 to 82 with each group's baseline (day 30) tumor volume. A two-sample t-test for each time point was used and compared the tumor volume between treated and control animals. To satisfy normality assumption for t-test, normalized tumor volume is log-transformed. For multiple comparisons in the osteosarcoma study, the inventors modeled counts of macro-metastatic lesions as a function of treatment group using a negative binomial generalized linear model (NBGLM). Treatment group comparisons were performed using model-based linear contrasts. All analyses were performed using R version 3.2.3. The inventors summarized the incidence rate ratios (IRRs) and corresponding 95% confidence intervals (CIs) comparing IgG1, hSFRP2 mAb and nivolumab to the combination of hSFRP2 mAb and nivolumab. The inventors envisioned that combination therapy would reduce the incidence of macro-metastatic lesions relative to single agent therapy, and therefore constructed IRRs with the treatment (IgG1, hSFRP2 mAb or nivolumab) represented in the denominator, facilitating interpretation of the impact of combination therapy relative to single agent.

### REFERENCES

1. Kawano Y, Kypta R. Secreted antagonists of the Wnt signalling pathway 1. J Cell Sci. 2003;116(Pt 13):2627-34.
2. Yamamura S, et al. Oncogenic functions of secreted Frizzled-related protein 2 in human renal cancer. MolCancer Ther. 2010;9(6):1680-7.
3. Esteve P, et al..Secreted frizzled-related proteins are required for Wnt/beta-catenin signalling activation in the vertebrate optic cup 1. Development. 2011;138(19):4179-84.
4. Gehmert S, Sadat S, Song Y H, Yan Y, Alt E. The anti-apoptotic effect of IGF-1 on tissue resident stem cells is mediated via PI3-kinase dependent secreted frizzled related protein 2 (Sfrp2) release. BiochemBiophysRes Commun. 2008;371(4):752-5.
5. Lee J L, Chang C J, Wu S Y, Sargan D R, Lin C T. Secreted frizzled-related protein 2 (SFRP2) is highly expressed in canine mammary gland tumors but not in normal mammary glands. Breast Cancer Res Treat. 2004;84(2):139-49.
6. Melkonyan H S, Chang W C, Shapiro J P, Mahadevappa M, Fitzpatrick P A, Kiefer M C, Tomei L D, Umansky S R. SARPs: a family of secreted apoptosis-related proteins. Proc NatlAcadSciUSA. 1997;94(25):13636-41.
7. Mirotsou M, Zhang Z, Deb A, Zhang L, Gnecchi M, Noiseux N, Mu H, Pachori A, Dzau V. Secreted frizzled related protein 2 (Sfrp2) is the key Akt-mesenchymal stem cell-released paracrine factor mediating myocardial survival and repair. Proc NatlAcadSciUSA. 2007; 104(5): 1643-8.
8. Gene Expression Omnibus [www.ncbi.nlm.nih.gov/geo/].
9. Bhati R, et al. Molecular characterization of human breast tumor vascular cells. Am J Pathol . 2008; 172(5): 1381-90.
10. Fontenot E, et al. A Novel Monoclonal Antibody to Secreted Frizzled Related Protein 2 Inhibits Tumor Growth. Molecular Cancer Therapeutics. 2013.
11. Lee J L, Chang C J, Chueh L L, Lin C T. Secreted Frizzled Related Protein 2 (sFRP2) Decreases Susceptibility to UV-Induced Apoptosis in Primary Culture of Canine Mammary Gland Tumors by NF-kappaB Activation or JNK Suppression. Breast Cancer Res Treat. 2006;100(1):49-58.
12. Techavichit P, Gao Y, Kurenbekova L, Shuck R, Donehower L A, Yustein J T. Secreted Frizzled-Related Protein 2 (sFRP2) promotes osteosarcoma invasion and metastatic potential. BMC Cancer. 2016;16(1):869. doi: 10.1186/s12885-016-2909-6. PubMed PMID: 27821163; PMCID: PMC5100268.
13. Singh S, et al. Impaired Wnt signaling in embryonal rhabdomyosarcoma cells from p53/c-fos double mutant mice. The American journal of pathology. 2010;177(4):2055-66. doi: 10.2353/ajpath.2010.091195. PubMed PMID: 20829439; PMCID: PMC2947299.
14. Tanaka M, Homme M, Yamazaki Y, Shimizu R, Takazawa Y, Nakamura T. Modeling Alveolar Soft Part Sarcoma Unveils Novel Mechanisms of Metastasis. Cancer Res. 2017;77(4):897-907. doi: 10.1158/0008-5472.CAN-16-2486. PubMed PMID: 27979841.
15. Roth W, Wild-Bode C, Platten M, Grimmel C, Melkonyan H S, Dichgans J, Weller M. Secreted Frizzled-related proteins inhibit motility and promote growth of human malignant glioma cells. Oncogene. 2000;19(37):4210-20.
16. Oshima T, Abe M, Asano J, Hara T, Kitazoe K, Sekimoto E, Tanaka Y, Shibata H, Hashimoto T, Ozaki S, Kido S, Inoue D, Matsumoto T. Myeloma cells suppress bone formation by secreting a soluble Wnt inhibitor, sFRP-2. Blood. 2005;106(9):3160-5. doi: 10.1182/blood-2004-12-4940. PubMed PMID: 16030194.
17. Sun Y, Zhu D, Chen F, Qian M, Wei H, Chen W, Xu J. SFRP2 augments WNT16B signaling to promote therapeutic resistance in the damaged tumor microenvironment. Oncogene. 2016;35(33):4321-34. doi: 10.1038/onc.2015.494. PubMed PMID: 26751775; PMCID: 4994019.
18. Xiao X, Xiao Y, Wen R, Zhang Y, Li X, Wang H, Huang J, Liu J, Long T, Tang J. Promoting roles of the secreted frizzled-related protein 2 as a Wnt agonist in lung cancer cells. Oncol Rep. 2015;34(5):2259-66. doi: 10.3892/or.2015.4221. PubMed PMID: 26323494; PMCID: 4583535.
19. Kaur A, et al. sFRP2 in the aged microenvironment drives melanoma metastasis and therapy resistance. Nature. 2016;532(7598):250-4. doi: 10.1038/nature17392. PubMed PMID: 27042933; PMCID: PMC4833579.
20. Tsuruta JK, Klauber-DeMore N, Streeter J, Samples J, Patterson C, Mumper R J, Ketelsen D, Dayton P. Ultrasound molecular imaging of secreted frizzled related protein-2 expression in murine angiosarcoma. PloS one. 2014;9(1):e86642. doi: 10.1371/journal.pone.0086642. PubMed PMID: 24489757; PMCID: 3906081.
21. Fontenot E, et al. A novel monoclonal antibody to secreted frizzled-related protein 2 inhibits tumor growth. MolCancer Ther. 2013;12(5):685-95. doi: 1535-7163.MCT-12-1066 [pii];10.1158/1535-7163.MCT-12-1066 [doi].
22. Siamakpour-Reihani S, Caster J, Bandhu N D, Courtwright A, Hilliard E, Usary J, Ketelsen D, Darr D, Shen X J, Patterson C, Klauber-DeMore N. The Role of Calcineurin/NFAT in SFRP2 Induced Angiogenesis-A Rationale for Breast Cancer Treatment with the Calcineurin Inhibitor Tacrolimus. PLoSOne. 2011;6(6):e20412.
23. Peterson Y K, Nasarre P, Bonilla I V, Hilliard E, Samples J, Morinelli T A, Hill E G, Klauber-DeMore N. Frizzled-5: a high affinity receptor for secreted frizzled-related protein-2 activation of nuclear factor of activated T-cells c3 signaling to promote angiogenesis. Angiogenesis. 2017;20(4):615-28. doi: 10.1007/s10456-017-9574-5. PubMed PMID: 28840375; PMCID: PMC5675522.
24. Courtwright A, Siamakpour-Reihani S, Arbiser J L, Banet N, Hilliard E, Fried L, Livasy C, Ketelsen D, Nepal D B, Perou CM, Patterson C, Klauber-DeMore N. Secreted Frizzle-Related Protein 2 Stimulates Angiogenesis via a Calcineurin/NFAT Signaling Pathway. Cancer Res. 2009.
25. Mancini M, Toker A. NFAT proteins: emerging roles in cancer progression. Nat Rev Cancer. 2009;9(11):810-20. doi: 10.1038/nrc2735. PubMed PMID: 19851316; PMCID: PMC2866640.
26. Macian F. NFAT proteins: key regulators of T-cell development and function. Nat Rev Immunol. 2005;5(6):472-84. doi: 10.1038/nri1632. PubMed PMID: 15928679.
27. Urso K, Alfranca A, Martinez-Martinez S, Escolano A, Ortega I, Rodriguez A, Redondo J M. NFATc3 regulates the transcription of genes involved in T-cell activation and angiogenesis. Blood. 2011;118(3):795-803. doi: 10.1182/blood-2010-12-322701. PubMed PMID: 21642596.
28. Dutta D, Barr V A, Akpan I, Mittelstadt P R, Singha L I, Samelson L E, Ashwell J D. Recruitment of calcineurin to the TCR positively regulates T cell activation. Nat Immunol. 2017;18(2):196-204. doi: 10.1038/ni.3640. PubMed PMID: 27941787.
29. Lindsey B A, Markel J E, Kleinerman E S. Osteosarcoma Overview. Rheumatol Ther. 2017;4(1):25-43. doi: 10.1007/s40744-016-0050-2. PubMed PMID: 27933467; PMCID: PMC5443719.
30. Tawbi H A, et al. Pembrolizumab in advanced soft-tissue sarcoma and bone sarcoma (SARC028): a multicentre, two-cohort, single-arm, open-label, phase 2 trial. Lancet Oncol. 2017;18(11):1493-501. doi: 10.1016/S1470-2045(17)30624-1. PubMed PMID: 28988646.
31. Wedekind M F, Wagner L M, Cripe T P. Immunotherapy for osteosarcoma: Where do we go from here? Pediatr Blood Cancer. 2018;65(9):e27227. doi: 10.1002/pbc.27227. PubMed PMID: 29923370.
32. Zhao S, et al. NKD2, a negative regulator of Wnt signaling, suppresses tumor growth and metastasis in osteosarcoma. Oncogene. 2015;34(39):5069-79. doi: 10.1038/onc.2014.429. PubMed PMID: 25579177; PMCID: PMC4802362.
33. Kim H, Yoo S, Zhou R, Xu A, Bernitz J M, Yuan Y, Gomes A M, Daniel M G, Su J, Demicco E G, Zhu J, Moore K A, Lee D F, Lemischka I R, Schaniel C. Oncogenic role of SFRP2 in p53-mutant osteosarcoma development via autocrine and paracrine mechanism. Proc Natl Acad Sci U S A. 2018;115(47):E11128-E37. doi: 10.1073/pnas.1814044115. PubMed PMID: 30385632; PMCID: PMC6255152.
34. Schoffski P, Cornillie J, Wozniak A, Li H, Hompes D. Soft tissue sarcoma: an update on systemic treatment options for patients with advanced disease. Oncol Res Treat. 2014;37(6):355-62. doi: 10.1159/000362631. PubMed PMID: 24903768.
35. Paoluzzi L, Cacavio A, Ghesani M, Karambelkar A, Rapkiewicz A, Weber J, Rosen G. Response to anti-PD1 therapy with nivolumab in metastatic sarcomas. Clin Sarcoma Res. 2016;6:24. doi: 10.1186/s13569-016-0064-0. PubMed PMID: 28042471; PMCID: PMC5200964.
36. Zhang J, et al. Cyclin D-CDK4 kinase destabilizes PD-L1 via cullin 3-SPOP to control cancer immune surveillance. Nature. 2018;553(7686):91-5. doi: 10.1038/nature25015. PubMed PMID: 29160310; PMCID: PMC5754234.
37. Bally A P, Lu P, Tang Y, Austin J W, Scharer C D, Ahmed R, Boss J M. N F-kappaB regulates PD-1 expression in macrophages. J Immunol. 2015;194(9):4545-54. doi: 10.4049/jimmunol.1402550. PubMed PMID: 25810391; PMCID: PMC4402259.
38. Oestreich K J, Yoon H, Ahmed R, Boss J M. NFATcl regulates PD-1 expression upon T cell activation. J Immunol. 2008;181(7):4832-9. PubMed PMID: 18802087; PMCID: PMC2645436.
39. Chatterjee S, et al. CD38-NAD(+)Axis Regulates Immunotherapeutic Anti-Tumor T Cell Response. Cell Metab. 2018;27(1):85-100 e8. doi: 10.1016/j.cmet.2017.10.006. PubMed PMID: 29129787; PMCID: PMC5837048.
40. Philip M, et al.. Chromatin states define tumour-specific T cell dysfunction and reprogramming. Nature. 2017;545(7655):452-6. doi: 10.1038/nature22367. PubMed PMID: 28514453; PMCID: PMC5693219.
41. Chen L, et al. CD38-Mediated Immunosuppression as a Mechanism of Tumor Cell Escape from PD-1/PD-L1 Blockade. Cancer Discov. 2018. doi: 10.1158/2159-8290.CD-17-1033. PubMed PMID: 30012853.
42. Vukcevic M, Zorzato F, Spagnoli G, Treves S. Frequent calcium oscillations lead to NFAT activation in human immature dendritic cells. J Biol Chem. 2010;285(21):16003-11. doi: 10.1074/jbc.M109.066704. PubMed PMID: 20348098; PMCID: PMC2871469.
43. Marklin M, Bugl, S., Bechtel, M., Poljak, A., Kopp, H., Kanz, L., Rao, A., Wirths, S., Muller, M. Ca2+/NFAT Signaling REgulates the Expression of CD38 and ZAP70 in Murine B Cells and Controls B1 a Cell Homeostasis. Blood. 2011;118(21):183.
44. Guidance for Industry. In vivo drug metabolism/drug interaction studies-study design, data analysis, and recommendations for dosing and labeling, U.S. Dept. Health and Human Svcs., FDA, Ctr. for Drug Eval. and Res., Ctr. For Biologics Eval. and Res., Clin./Pharm., November 1999<http://www.fda.gov/cber/gdlns/metabol.pdf>.
45. Kleinschmidt-DeMasters et al. (2005) New England Journal of Medicine, 353:369-379.
46. Langer-Gould et al. (2005) New England Journal of Medicine, 353:369-379.
47. Rudick et al. (2006) New England Journal of Medicine, 354:911-923
48. Salama, Hassan H., et al. “Effects of Combination Therapy of Beta-Interferon 1a and Prednisone on Serum Immunologic Markers in Patients with Multiple Sclerosis.” Multiple Sclerosis, vol. 9, no. 1, February 2003, pp. 28-31. PubMed.
49. Brod et al. (2000) Annals of Neurology, 47:127-131.
50. U.S. Pat. Nos. 8,734,789, and 9,073,982

It is to be understood that the invention is not limited to the particular embodiments of the invention described above, as variations of the particular embodiments may be made and still fall within the scope of the appended claims.

The invention will be further described, without limitation, by the following numbered paragraphs:
1. A pharmaceutical combination, comprising a therapeutically effective amount of a SFRP2 antagonist, CD38 antagonist, and/or PD-1 antagonist and a therapeutically effective amount of a PD-1 antagonist.
2. The pharmaceutical combination according to paragraph 1, wherein the SFRP2, CD38, and/or PD-1 antagonist is:
   a. an antibody, or antigen binding fragment of an antibody, that specifically binds to, and inhibits activation of, an SFRP2, CD38, and/or PD-1 receptor, or
   b. a soluble form of an SFRP2, CD38, and/or PD-1 receptor that specifically binds to a SFRP2, CD38, and/or PD-1 ligand and inhibits the SFRP2, CD38, and/or PD-1 ligand from binding to the SFRP2, CD38, and/or PD-1 receptor.
3. The pharmaceutical combination according to any one of paragraphs 1-2, wherein the SFRP2, CD38, and/or PD-1 antagonist is a SFRP2, CD38, and/or PD-1 monoclonal antibody (mAb).
4. The pharmaceutical combination according to paragraph 3, wherein the SFRP2 monoclonal antibody is human or humanized.
5. The pharmaceutical combination according to any one of paragraphs 1-4, wherein the PD-1 antagonist is:
   a. an antibody, or antigen binding fragment of an antibody, that specifically binds to, and inhibits activation of, an PD-1 receptor, or
   b. a soluble form of an PD-1 receptor that specifically binds to a PD-1 ligand and inhibits the PD-1 ligand from binding to the PD-1 receptor.
6. The pharmaceutical combination according to paragraph 5, wherein the PD-1 antagonist is the soluble form of an PD-1 receptor and the PD-1 ligand is PD-L1 or PD-L2.
7. The pharmaceutical combination according to any one of paragraphs 1-5, wherein the PD-1 antagonist is a PD-1 monoclonal antibody.
8. The pharmaceutical combination according to any one of paragraphs 1-5, wherein the PD-1 antagonist is nivolumab.
9. The pharmaceutical combination according to any one of paragraphs 1-5, wherein the PD-1 antagonist is pembrolizumab, avelumab, durvalumab, cemiplimab, or atezolizumab.
10. The pharmaceutical combination according to any one of paragraphs 1-9, wherein the therapeutically effective amount of SFRP2, CD38, and/or PD-1 antagonist is 0.1 mg/kg body weight to 100 mg/kg body weight.
11. The pharmaceutical combination according to any one of paragraphs 1-9, wherein the therapeutically effective amount of SFRP2, CD38, and/or PD-1 antagonist is 0.2-3, 0.27-2.70, 0.27, 0.54, 1.35, or 2.70 mg per kg body weight.
12. The pharmaceutical combination according to any one of paragraphs 1-11, wherein the therapeutically effective amount of SFRP2, CD38, and/or PD-1 antagonist is 10 mg-200 mg, 17 mg, 33 mg, 84 mg, or 167 mg
13. The pharmaceutical combination according to any one of paragraphs 1-12, wherein the therapeutically effective amount of PD-1 antagonist is 0.1 mg/kg body weight to 100 mg/kg body weight.
14. The pharmaceutical combination according to any one of paragraphs 1-12, wherein the therapeutically effective amount of PD-1 antagonist is 0.02-1.2, 0.027-1.08, 0.027 or 1.08 mg/kg body weight.
15. The pharmaceutical combination according to any one of paragraphs 1-14, wherein the therapeutically effective amount of PD-1 antagonist is 1-80, 1.6-67, 1.6 or 67 mg/kg body weight.
16. A method for the treatment of cancer, comprising administering a therapeutically effective amount of a SFRP2, CD38, and/or PD-1 antagonist and a therapeutically effective amount of an PD-1 antagonist to a subject in need thereof.
17. The method according to paragraph 16, wherein the administration is simultaneous or sequential.
18. The method according to paragraph 16 or 17, wherein the SFRP2, CD38, and/or PD-1 antagonist is:
   a. an antibody, or antigen binding fragment of an antibody, that specifically binds to, and inhibits activation of, an SFRP2, CD38, and/or PD-1 receptor, or
   b. a soluble form of an SFRP2, CD38, and/or PD-1 receptor that specifically binds to a SFRP2, CD38, and/or PD-1 ligand and inhibits the SFRP2, CD38, and/or PD-1 ligand from binding to the SFRP2, CD38, and/or PD-1 receptor.
19. The method according to any one of paragraphs 16-18, wherein the SFRP2, CD38, and/or PD-1 antagonist is a SFRP2, CD38, and/or PD-1 monoclonal antibody (mAb).
20. The method according to paragraph 19, wherein the SFRP2 monoclonal antibody is human or humanized.
21. The method according to any one of paragraphs 16-20, wherein the PD-1 antagonist is:
   a. an antibody, or antigen binding fragment of an antibody, that specifically binds to, and inhibits activation of, an PD-1 receptor, or
   b. a soluble form of an PD-1 receptor that specifically binds to a PD-1 ligand and inhibits the PD-1 ligand from binding to the PD-1 receptor.
22. The method according to paragraph 21, wherein the PD-1 antagonist is the soluble form of the PD-1 receptor and the PD-1 ligand is PD-L1 or PD-L2.
23. The method according to any one of paragraphs 16-22, wherein the PD-1 antagonist is a PD-1 monoclonal antibody.
24. The method according to any one of paragraphs 16-23, wherein the PD-1 antagonist is nivolumab.
25. The method of any one of paragraphs 16-23, wherein the PD-1 antagonist is pembrolizumab, avelumab, durvalumab, cemiplimab, or atezolizumab.
26. The method according to any one of paragraphs 16-25, wherein said cancer is breast cancer.
27. The method according to any one of paragraphs 16-26, wherein said cancer is angiosarcoma, lung cancer, osteosarcoma, melanoma, non-small cell lung cancer, or kidney cancer.
28. The method according to any one of paragraphs 16-27, wherein the administration of the SFRP2, CD38, and/or PD-1 antagonist precedes the administration of the PD-1 antagonist.
29. The method according to any one of paragraphs 16-27, wherein the administration of the PD-1 antagonist precedes the administration of SFRP2, CD38, and/or PD-1 antagonist.
30. The method according to any one of paragraphs 16-29, wherein the SFRP2, CD38, and/or PD-1 antagonist is administered adjunctively to the PD-1 antagonist.
31. The method according to any one of paragraphs 16-29, wherein the PD-1 antagonist is administered adjunctively to the SFRP2, CD38, and/or PD-1 antagonist.
32. The method according to any one of paragraphs 16-31, wherein the SFRP2, CD38, and/or PD-1 antagonist is administered daily, more often than once daily or less often than once daily.
33. The method according to any one of paragraphs 16-31, wherein the SFRP2, CD38, and/or PD-1 antagonist is administered once every 3 days, once every week, once every 2 weeks, once every 3 weeks or once every 4 weeks.
34. The method of any one of paragraphs 16-33, wherein the PD-1 antagonist is administered daily, more often than once daily or less often than once daily.
35. The method according to any one of paragraphs 16-33, wherein the PD-1 antagonist is administered once every 3 days, once every week, once every 2 weeks, once every 3 weeks or once every 4 weeks.
36. The method according to any one of paragraphs 16-35, wherein the PD-1 antagonist is nivolumab and the amount of the nivolumab administered to the subject is 3 mg/kg body weight every 3 weeks, 240 mg every 2 weeks or 480 mg every 4 weeks.
37. The method according to any one of paragraphs 16-35, wherein the PD-1 antagonist is pembrolizumab and the amount of the pembrolizumab administered to the subject is 200 mg every 3 weeks.
38. The method according to any one of paragraphs 16-35, wherein the PD-1 antagonist is avelumab and the amount of the avelumab administered to the subject is 800 mg every 2 weeks.
39. The method according to any one of paragraphs 16-35, wherein the PD-1 antagonist is durvalumab and the amount of the durvalumab administered to the subject is 10 mg/kg body weight every 2 weeks.
40. The method according to any one of paragraphs 16-35, wherein the PD-1 antagonist is cemiplimab and the amount of the cemiplimab administered to the subject is 250 mg every 3 weeks.
41. The method according to any one of paragraphs 16-35, wherein the PD-1 antagonist is atezolizumab and the amount of the atezolizumab administered to the subject is 840 mg every 2 weeks, 1200 mg every 3 weeks or 1680 mg every 4 weeks.
42. The method according to any one of paragraphs 16-41, wherein the subject is receiving PD-1 antagonist therapy prior to initiating SFRP2, CD38, and/or PD-1 antagonist therapy.
43. The method according to any one of paragraphs 16-41, wherein the subject is receiving SFRP2, CD38, and/or PD-1 antagonist therapy prior to initiating PD-1 antagonist therapy.
44. The method according to paragraph 42 or 43, where in the subject is receiving a first therapy for at least 8 weeks, at least 10 weeks, at least 24 weeks, at least 28 weeks, at least 48 weeks or at least 52 weeks prior to initiating a second therapy.
45. The method according to any one of paragraphs 16-44, wherein the periodic administration of the SFRP2, CD38, and/or PD-1 antagonist and/or the PD-1 antagonist continues for at least 3 days, for at least 30 days, for at least 42 days, for at least 8 weeks, for at least 12 weeks, for at least 24 weeks or for at least 6 months.
46. The method according to any one of paragraphs 16-45, wherein each of the amount of SFRP2, CD38, and/or PD-1 antagonist when taken alone, and the amount of PD-1 antagonist when taken alone is effective to treat the subject.
47. The method according to any one of paragraphs 16-45, wherein either the amount of SFRP2, CD38, and/or PD-1 antagonist when taken alone, the amount of PD-1 antagonist when taken alone, or each such amount when taken alone is not effective to treat the subject.
48. The method according to any one of paragraphs 16-45, wherein either the amount of SFRP2, CD38, and/or PD-1 antagonist when taken alone, the amount of PD-1 antagonist when taken alone, or each such amount when taken alone is less effective to treat the subject.
49. The method according to any one of paragraphs 16-48, wherein the subject is a human patient.
50. The method according to any one of paragraphs 16-49, wherein the patient previously received PD-1 antagonist therapy and ceased receiving PD-1 antagonist therapy prior to receiving the combination therapy.
51. The method according to any one of paragraphs 16-50, wherein the patient previously failed to respond to PD-1 antagonist therapy or the administration of PD-1 antagonist monotherapy failed to treat the subject.
52. A kit for treating a patient suffering from cancer, comprising a therapeutically effective amount of an SFRP2, CD38, and/or PD-1 antagonist, a therapeutically effective amount of an PD-1 antagonist, and an insert comprising instructions for use of the kit.
53. A pharmaceutical composition comprising an amount of an PD-1 antagonist and an amount of a SFRP2, CD38, and/or PD-1 antagonist.
54. The pharmaceutical composition according to paragraph 53, comprising essentially an amount of an PD-1 antagonist and an amount of a SFRP2, CD38, and/or PD-1 antagonist.
55. The pharmaceutical composition according to paragraph 53 or 54, for use in treating a subject afflicted with cancer, wherein the amount of the PD-1 antagonist and an amount of the SFRP2, CD38, and/or PD-1 antagonist are to be administered simultaneously, contemporaneously or concomitantly.
56. A therapeutic package for dispensing to, or for use in dispensing to, a subject afflicted with cancer, which comprises: a) one or more unit doses, each such unit dose comprising: i) amount of PD-1 antagonist and ii) an amount of SFRP2, CD38, and/or PD-1 antagonist wherein the respective amounts of said PD-1 antagonist and said SFRP2, CD38, and/or PD-1 antagonist in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.
57. A SFRP2, CD38, and/or PD-1 antagonist for use as an add-on therapy or in combination with an PD-1 antagonist in treating a subject afflicted with cancer.
58. An PD-1 antagonist for use as an add-on therapy or in combination with SFRP2, CD38, and/or PD-1 antagonist in treating a subject afflicted with cancer.
59. Use of an amount of SFRP2, CD38, and/or PD-1 antagonist and an amount of an PD-1 antagonist in the preparation of a combination for treating a subject afflicted with cancer wherein the SFRP2, CD38, and/or PD-1 antagonist and the PD-1 antagonist are prepared to be administered simultaneously, contemporaneously or concomitantly.
60. A combination of SFRP2, CD38, and/or PD-1 antagonist and an PD-1 antagonist for use in the manufacture of a medicament.
61. The combination according to paragraph 60, wherein the medicament is for the treatment, prevention, or alleviation of a symptom of cancer.
62. A method for the treatment of cancer, comprising administering a therapeutically effective amount of a SFRP2 monoclonal antibody (mAb) to a subject in need thereof, wherein the subject has increased expression of CD38 and/or PD-1.
63. The method of paragraph 62, wherein the subject's T-cells have increased expression of CD3 8 and/or PD-1.
64. The method according to paragraph 62 or 63, wherein the SFRP2 monoclonal antibody is human or humanized.
65. The method according to any one of paragraphs 62-64, wherein said cancer is breast cancer.
66. The method according to any one of paragraphs 62-64, wherein said cancer is angiosarcoma, lung cancer, osteosarcoma, melanoma, non-small cell lung cancer, or kidney cancer.
67. The method according to any one of paragraphs 62-66, wherein the SFRP2 monoclonal antibody is administered daily, more often than once daily or less often than once daily.
68. The method according to any one of paragraphs 62-67, wherein the SFRP2 monoclonal antibody is administered once every 3 days, once every week, once every 2 weeks, once every 3 weeks or once every 4 weeks.
69. The method according to any one of paragraphs 62-67, wherein the periodic administration of the SFRP2 monoclonal antibody continues for at least 3 days, for at least 30 days, for at least 42 days, for at least 8 weeks, for at least 12 weeks, for at least 24 weeks or for at least 6 months.
70. The method according to any one of paragraphs 62-69, wherein the subject is a human patient.

What is claimed is:

1. A method for the treatment of cancer, comprising administering a therapeutically effective amount of a SFRP2, CD38, and/or PD-1 antagonist and a therapeutically effective amount of an PD-1 antagonist to a subject in need thereof

2. The method according to claim 1, wherein the administration is simultaneous or sequential.

3. The method according to claim 1, wherein the SFRP2, CD38, and/or PD-1 antagonist is:
a. an antibody, or antigen binding fragment of an antibody, that specifically binds to, and inhibits activation of, an SFRP2, CD38, and/or PD-1 receptor, or
b. a soluble form of an SFRP2, CD38, and/or PD-1 receptor that specifically binds to a SFRP2 and/or CD38 ligand and inhibits the SFRP2, CD38, and/or PD-1 ligand from binding to the SFRP2, CD38, and/or PD-1 receptor.

4. The method according to claim 1, wherein the SFRP2, CD38, and/or PD-1 antagonist is a SFRP2, CD38, and/or PD-1 monoclonal antibody (mAb).

5. The method according to claim 4, wherein the SFRP2 monoclonal antibody is human or humanized.

6. The method according to claim 1, wherein the PD-1 antagonist is:
a. an antibody, or antigen binding fragment of an antibody, that specifically binds to, and inhibits activation of, an PD-1 receptor, or
b. a soluble form of an PD-1 receptor that specifically binds to a PD-1 ligand and inhibits the PD-1 ligand from binding to the PD-1 receptor.

7. The method according to claim 6, wherein the PD-1 ligand is PD-L1 or PD-L2.

8. The method according to claim 1, wherein the PD-1 antagonist is a PD-1 monoclonal antibody.

9. The method according to claim 1, wherein the PD-1 antagonist is nivolumab.

10. The method of claim 1, wherein the PD-1 antagonist is pembrolizumab, avelumab, durvalumab, cemiplimab, or atezolizumab.

11. The method according to claim 1, wherein said cancer is breast cancer.

12. The method according to claim 1, wherein said cancer is angiosarcoma, lung cancer, osteosarcoma, melanoma, non-small cell lung cancer, or kidney cancer.

13. The method according to claim 1, wherein the administration of the SFRP2, CD38, and/or PD-1 antagonist precedes the administration of the PD-1 antagonist.

14. The method according to claim 1, wherein the administration of the PD-1 antagonist precedes the administration of SFRP2, CD38, and/or PD-1 antagonist.

15. The method according to claim 1, wherein the SFRP2, CD38, and/or PD-1 antagonist is administered adjunctively to the PD-1 antagonist.

16. The method according to claim 1, wherein the PD-1 antagonist is administered adjunctively to the SFRP2, CD38, and/or PD-1 antagonist.

17. The method according to claim 1, wherein the SFRP2, CD38, and/or PD-1 antagonist is administered daily, more often than once daily or less often than once daily.

18. The method according to claim 1, wherein the SFRP2 antagonist is administered once every 3 days, once every week, once every 2 weeks, once every 3 weeks or once every 4 weeks.

19. The method of claim 1, wherein the PD-1 antagonist is administered daily, more often than once daily or less often than once daily.

20. The method according to claim 1, wherein the PD-1 antagonist is administered once every 3 days, once every week, once every 2 weeks, once every 3 weeks or once every 4 weeks.

21. The method according to claim 1, wherein the PD-1 antagonist is nivolumab and the amount of the nivolumab administered to the subject is 3 mg/kg body weight every 3 weeks, 240 mg every 2 weeks or 480 mg every 4 weeks.

22. The method according to claim 1, wherein the PD-1 antagonist is pembrolizumab and the amount of the pembrolizumab administered to the subject is 200 mg every 3 weeks.

23. The method according to claim 1, wherein the PD-1 antagonist is avelumab and the amount of the avelumab administered to the subject is 800 mg every 2 weeks.

24. The method according to claim 1, wherein the PD-1 antagonist is durvalumab and the amount of the durvalumab administered to the subject is 10 mg/kg body weight every 2 weeks.

25. The method according to claim 1, wherein the PD-1 antagonist is cemiplimab and the amount of the cemiplimab administered to the subject is 250 mg every 3 weeks.

26. The method according to claim 1, wherein the PD-1 antagonist is atezolizumab and the amount of the atezolizumab administered to the subject is 840 mg every 2 weeks, 1200 mg every 3 weeks or 1680 mg every 4 weeks.

27. The method according to claim 1, wherein the subject is receiving PD-1 antagonist therapy prior to initiating SFRP2, CD38, and/or PD-1 antagonist therapy.

28. The method according to claim 1, wherein the subject is receiving SFRP2, CD38, and/or PD-1 antagonist therapy prior to initiating PD-1 antagonist therapy.

29. The method according to claim 27 or 28, where in the subject is receiving a first therapy for at least 8 weeks, at least 10 weeks, at least 24 weeks, at least 28 weeks, at least 48 weeks or at least 52 weeks prior to initiating a second therapy.

30. The method according to claim 1, wherein the periodic administration of the SFRP2, CD38, and/or PD-1 antagonist and/or the PD-1 antagonist continues for at least 3 days, for at least 30 days, for at least 42 days, for at least 8 weeks, for at least 12 weeks, for at least 24 weeks or for at least 6 months.

31. The method according to claim 1, wherein each of the amount of SFRP2, CD38, and/or PD-1 antagonist when taken alone, and the amount of PD-1 antagonist when taken alone is effective to treat the subject.

32. The method according to claim 1, wherein either the amount of SFRP2, CD38, and/or PD-1 antagonist when taken alone, the amount of PD-1 antagonist when taken alone, or each such amount when taken alone is not effective to treat the subject.

33. The method according to claim 1, wherein either the amount of SFRP2, CD38, and/or PD-1 antagonist when taken alone, the amount of PD-1 antagonist when taken alone, or each such amount when taken alone is less effective to treat the subject.

34. The method according to claim 1, wherein the subject is a human patient.

35. The method according to claim 1, wherein the patient previously received PD-1 antagonist therapy and ceased receiving PD-1 antagonist therapy prior to the combination therapy.

36. The method according to claim 35, wherein the patient previously failed to respond to PD-1 antagonist therapy or the PD-1 antagonist failed to treat the subject.

37. A kit for treating a patient suffering from cancer, comprising a therapeutically effective amount of an SFRP2, CD38, and/or PD-1 antagonist, a therapeutically effective amount of an PD-1 antagonist, and an insert comprising instructions for use of the kit.

38. A pharmaceutical composition comprising an amount of an PD-1 antagonist and an amount of a SFRP2, CD38, and/or PD-1 antagonist.

39. The pharmaceutical composition according to claim 38, comprising essentially an amount of an PD-1 antagonist and an amount of a SFRP2, CD38, and/or PD-1 antagonist.

40. The pharmaceutical composition according to claim 38, for use in treating a subject afflicted with cancer, wherein the amount of the PD-1 antagonist and an amount of the SFRP2, CD38, and/or PD-1 antagonist are to be administered simultaneously, contemporaneously or concomitantly.

41. A therapeutic package for dispensing to, or for use in dispensing to, a subject afflicted with cancer, which comprises: a) one or more unit doses, each such unit dose comprising: i) amount of PD-1 antagonist and ii) an amount of SFRP2, CD38, and/or PD-1 antagonist wherein the respective amounts of said PD-1 antagonist and said SFRP2, CD38, and/or PD-1 antagonist in said unit dose are effective, upon concomitant administration to said subject, to treat the subject, and b) a finished pharmaceutical container therefor, said container containing said unit dose or unit doses, said container further containing or comprising labeling directing the use of said package in the treatment of said subject.

42. A SFRP2, CD38, and/or PD-1 antagonist for use as an add-on therapy or in combination with an PD-1 antagonist in treating a subject afflicted with cancer.

43. An PD-1 antagonist for use as an add-on therapy or in combination with SFRP2, CD38, and/or PD-1 antagonist in treating a subject afflicted with cancer.

44. Use of an amount of SFRP2, CD38, and/or PD-1 antagonist and an amount of an PD-1 antagonist in the preparation of a combination for treating a subject afflicted with cancer wherein the SFRP2, CD38, and/or PD-1 antagonist and the PD-1 antagonist are prepared to be administered simultaneously, contemporaneously or concomitantly.

45. A combination of SFRP2, CD38, and/or PD-1 antagonist and an PD-1 antagonist for use in the manufacture of a medicament.

46. The combination according to claim 45, wherein the medicament is for the treatment, prevention, or alleviation of a symptom of cancer.

47. A pharmaceutical combination, comprising a therapeutically effective amount of a SFRP2, CD38, and/or PD-1 antagonist and a therapeutically effective amount of a PD-1 antagonist.

48. The pharmaceutical combination according to claim 47, wherein the SFRP2, CD38, and/or PD-1 antagonist is:
a. an antibody, or antigen binding fragment of an antibody, that specifically binds to, and inhibits activation of, an SFRP2, CD38, and/or PD-1 receptor, or
b. a soluble form of an SFRP2, CD38, and/or PD-1 receptor that specifically binds to a SFRP2, CD38, and/or PD-1 ligand and inhibits the SFRP2, CD38, and/or PD-1 ligand from binding to the SFRP2, CD38, and/or PD-1 receptor.

49. The pharmaceutical combination according to claim 47, wherein the SFRP2, CD38, and/or PD-1 antagonist is a SFRP2, CD38, and/or PD-1 monoclonal antibody (mAb).

50. The pharmaceutical combination according to claim 47, wherein the SFRP2 monoclonal antibody is human or humanized.

51. The pharmaceutical combination according to claim 47, wherein the PD-1 antagonist is:
a. an antibody, or antigen binding fragment of an antibody, that specifically binds to, and inhibits activation of, an PD-1 receptor, or
b. a soluble form of an PD-1 receptor that specifically binds to a PD-1 ligand and inhibits the PD-1 ligand from binding to the PD-1 receptor.

52. The pharmaceutical combination according to claim 51, wherein the PD-1 ligand is PD-L1 or PD-L2.

53. The pharmaceutical combination according to claim 47, wherein the PD-1 antagonist is a PD-1 monoclonal antibody.

54. The pharmaceutical combination according to claim 47, wherein the PD-1 antagonist is nivolumab.

55. The pharmaceutical combination according to claim 47, wherein the PD-1 antagonist is pembrolizumab, avelumab, durvalumab, cemiplimab, or atezolizumab.

56. The pharmaceutical combination according to claim 47, wherein the therapeutically effective amount of SFRP2, CD38, and/or PD-1 antagonist is 0.1 mg/kg body weight to 100 mg/kg body weight.

57. The pharmaceutical combination according to claim 47, wherein the therapeutically effective amount of SFRP2, CD38, and/or PD-1 antagonist is 0.2-3, 0.27-2.70, 0.27, 0.54, 1.35, or 2.70 mg per kg body weight.

58. The pharmaceutical combination according to claim 47, wherein the therapeutically effective amount of SFRP2, CD38, and/or PD-1 antagonist is 10 mg-200 mg, 17 mg, 33 mg, 84 mg, or 167 mg

59. The pharmaceutical combination according to claim 47, wherein the therapeutically effective amount of PD-1 antagonist is 0.1 mg/kg body weight to 100 mg/kg body weight.

60. The pharmaceutical combination according to claim 47, wherein the therapeutically effective amount of PD-1 antagonist is 0.02-1.2, 0.027-1.08, 0.027 or 1.08 mg/kg body weight.

61. The pharmaceutical combination according to claim 47, wherein the therapeutically effective amount of PD-1 antagonist is 1-80, 1.6-67, 1.6 or 67 mg/kg body weight.

62. A method for the treatment of cancer, comprising administering a therapeutically effective amount of a SFRP2 monoclonal antibody (mAb) to a subject in need thereof, wherein the subject has increased expression of CD38 and/or PD-1.

63. The method of claim 62, wherein the subject's T-cells have increased expression of CD38 and/or PD-1.

64. The method according to claim 62, wherein the SFRP2 monoclonal antibody is human or humanized.

65. The method according to claim 62, wherein said cancer is breast cancer, angiosarcoma, lung cancer, osteosarcoma, melanoma, non-small cell lung cancer, or kidney cancer.

66. The method according to claim 62, wherein the SFRP2 monoclonal antibody is administered daily, more often than once daily or less often than once daily.

67. The method according to claim 62, wherein the SFRP2 monoclonal antibody is administered once every 3 days, once every week, once every 2 weeks, once every 3 weeks or once every 4 weeks.

68. The method according to claim 62, wherein the periodic administration of the SFRP2 monoclonal antibody continues for at least 3 days, for at least 30 days, for at least 42 days, for at least 8 weeks, for at least 12 weeks, for at least 24 weeks or for at least 6 months.

69. The method according to claim 62, wherein the subject is a human patient.
